(19) United States
(12) Patent Application Publication
(10) Pub. No.: US 2024/0316089 A1
(43) Pub. Date: Sep. 26, 2024

(54) COMBINATIONS FOR THE TREATMENT OF NEUROLOGICAL CONDITIONS

(71) Applicant: KetaMed MSO, LLC, Bedford Hills, NY (US)

(72) Inventor: Douglas Kornreich, Ridgefield, CT (US)

(21) Appl. No.: 18/614,320

(22) Filed: Mar. 22, 2024

Related U.S. Application Data
(60) Provisional application No. 63/453,933, filed on Mar. 22, 2023.

(51) Int. Cl. A61K 31/7076; A61K 9/00; A61K 31/135

(52) U.S. Cl. CPC A61K 31/7076; A61K 9/0019; A61K 9/0053; A61K 31/135

(57) ABSTRACT

The present disclosure relates to pharmaceutical combinations comprising S-adenosylmethionine (SAM-e) and an N-methyl-D-aspartate (NMDA) receptor antagonist e.g., ketamine or a pharmaceutically acceptable salt thereof. The present disclosure further relates to methods of treatment and the use of such compositions in the treatment of neurological conditions, e.g., treatment resistant depression and pain.

### CROSS-REFERENCE TO RELATED PATENT APPLICATIONS

This application claims benefit of U.S. Provisional Patent Application No. 63/453,933, filed on Mar. 22, 2023, which is incorporated by reference in its entirety.

### FIELD

The present disclosure relates to pharmaceutical combinations comprising S-adenosylmethionine (SAM-e) and an N-methyl-D-aspartate (NMDA) receptor antagonist e.g., ketamine or salt thereof. The present disclosure further relates to methods of treatment and the use of such compositions in the treatment of neurological conditions, e.g., treatment resistant depression and pain.

### BACKGROUND

Ketamine, an N-methyl-D-aspartate (NMDA) receptor antagonist, is an effective, fast-acting therapeutic intervention for patients with treatment-resistant depression. Treatment with ketamine is associated with increased blood pressure (hypertension), requiring continuous monitoring during infusion. About 30% of patients require further intervention with antihypertensive medication during ketamine administration. Additionally, treatment with ketamine is associated with hepatobiliary adverse events, and ketamine-induced liver injury has been reported. Moreover, treatment with ketamine can cause transient dissociation, a temporary mental state in which a person feels ‘detached’, or disconnected from their emotions, surroundings, and sense of identity, i.e., the person becomes less aware of what is actually around them and starts to feel disconnected from their body. A patient's intolerance of dissociation may prevent the administration of an adequate therapeutic dose of NMDAR antagonist.

S-adenosylmethionine is required for the synthesis of monoamine neurotransmitters such as norepinephrine (NE), dopamine (DA) and serotonin (5-hydroxytryptophan (5HT)), which play a role in maintaining normal mood.

### SUMMARY

In accordance with the illustrative embodiments provided herein, the present disclosure relates to pharmaceutical combinations comprising an NMDA receptor antagonist and S-adenosylmethionine, methods of treatment and uses utilizing the pharmaceutical combinations for the treatment of neurological disorders.

In some embodiments, the present disclosure provides a pharmaceutical combination comprising: (i) a first pharmaceutical composition comprising in a unit dosage form an N-methyl-D-aspartate (NMDA) receptor antagonist or a pharmaceutically acceptable salt thereof and a pharmaceutically acceptable carrier or excipient; and (ii) a second pharmaceutical composition comprising in a solid unit dosage form S-adenosylmethionine or a pharmaceutically acceptable salt thereof, and a pharmaceutically acceptable carrier or excipient.

In some embodiments, the present disclosure provides a fixed dose combination comprising, in unit dosage form: (i) an N-methyl-D-aspartate (NMDA) receptor antagonist or a pharmaceutically acceptable salt thereof; (ii) S-adenosylmethionine or a pharmaceutically acceptable salt thereof; and (iii) and a pharmaceutically acceptable carrier or excipient.

In some embodiments, the present disclosure provides a method of treating a neurological condition in a subject in need thereof, the method comprising administering to the subject a combination comprising a therapeutically effective amount of an N-methyl-D-aspartate (NMDA) receptor antagonist or a pharmaceutically acceptable salt thereof, and a therapeutically effective amount of S-adenosylmethionine or a pharmaceutically acceptable salt thereof.

In some embodiments, the NMDA receptor antagonist is ketamine (including racemic ketamine, (S)-ketamine (Esketamine), and (R)-ketamine (Arketamine)), or a pharmaceutically acceptable salt thereof.

Other aspects and advantages of the present disclosure are described further in the following detailed description.

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the disclosure and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term.

The term “combination” or “pharmaceutical combination” as used herein interchangeably, refers to a combination comprising (i) an N-methyl-D-aspartate (NMDA) receptor antagonist (e.g., ketamine) or a pharmaceutically acceptable salt thereof; and (ii) S-adenosylmethionine (SAM-e) or a pharmaceutically acceptable salt thereof. In some embodiments, the NMDA receptor antagonist (e.g., ketamine or pharmaceutically acceptable salt thereof) and the SAM-e or pharmaceutically acceptable salt thereof are provided in separate pharmaceutical compositions. In some embodiments, the NMDA receptor antagonist (e.g., ketamine or pharmaceutically acceptable salt thereof) and the SAM-e or pharmaceutically acceptable salt thereof are provided in one pharmaceutical composition comprising both active ingredients, i.e., a fixed dose combination (FDC).

The term “patient” as used in this application means a human subject.

The term “in need thereof” would be a subject known or suspected of having at least one condition as described herein.

The terms “treat”, “treating” or “treatment” of a state, disorder or condition includes: (1) preventing or delaying the appearance of clinical symptoms of the state, disorder, or condition developing in a person who may be afflicted with or predisposed to the state, disorder or condition but does not yet experience or display clinical symptoms of the state, disorder or condition; or (2) inhibiting the state, disorder or condition, i.e., arresting, reducing or delaying the development of the disease or a relapse thereof (in case of maintenance treatment) or at least one clinical symptom, sign, or test, thereof; or (3) relieving the disease, i.e., causing regression of the state, disorder or condition or at least one of its clinical or sub-clinical symptoms or signs.

The term “agent” as used herein means a substance that produces or is capable of producing an effect and would include, but is not limited to, chemicals, pharmaceuticals, biologics, small organic molecules, antibodies, nucleic acids, peptides, and proteins. The terms “agent,” “compound” and “drug” are interchangeable.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 10%, more preferably up to 5%, and more preferably still up to 1% of a given value. Where particular values are described in the application and claims, unless otherwise stated, the term “about” meaning within an acceptable error range for the particular value should be assumed.

As used herein, the terms “therapeutically effective amount”, “therapeutically effective dose” and “effective amount” refer to an amount of the compound and composition which is sufficient to effect beneficial or desired results, that, when administered as monotherapy or in combination with an additional therapeutic agent to a cell, tissue, or subject, is effective to cause a measurable improvement in one or more symptoms of a condition or in delaying, reducing or mitigating the progression of such condition, or maintaining therapeutic efficacy while ameliorating or reducing side effects or providing another benefit to the subject. For example, in reference to combination therapy, “therapeutically effective amount”, “therapeutically effective dose” and “effective amount” can also refer to an amount that is effective to cause a measurable improvement of one or more side effects associated with one or more components of the combination. For example, by way of illustration, for a combination of an NMDA receptor antagonist and SAM-e, the term therapeutically effective amount can mean an amount that is effective to cause a measurable improvement in hemodynamic stability, hepatoprotectivity, decreased dissociation, or other adverse effects when compared with monotherapy of an N-methyl-D-aspartate (NMDA) receptor antagonist or a pharmaceutically acceptable salt thereof. A therapeutically effective dose further refers to that amount of the compound sufficient to result in at least partial amelioration of symptoms of a medical condition, e.g., reducing, healing, prevention or amelioration of symptoms of the medical condition. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, sequentially in any order, or simultaneously. A combined therapeutic effect can be “additive” (i.e., the combined effects of two drugs equal the sum of the effects of the two drugs acting independently). Alternatively, the therapeutic response is the same or similar to the therapeutic effect of either of the two drugs, but the adverse effects of one or more drugs is reduced as compared with the adverse effect of that drug administered in monotherapy. A combined therapeutic effect can also be “synergistic” (i.e., the combined effects of two drugs is greater than the sum of the effects of the two drugs acting independently). An effective amount can also result in an improvement in a subjective measure in cases where subjective measures are used to assess disease severity.

The phrase “pharmaceutically-acceptable” or “pharmacologically acceptable” as used herein refers to compounds and compositions that are physiologically tolerable and do not typically produce an allergic or similar untoward reaction, such as, without limitation, gastric upset, headache and dizziness, when administered to a human. The term “pharmaceutically acceptable” or “pharmacologically acceptable” can also refer to compounds and compositions that are approved by a regulatory agency of a government or listed in the U.S. or EP Pharmacopeia or other generally recognized pharmacopeia for use in animals, and more particularly in humans.

### Combinations

In some embodiments, the present disclosure provides a pharmaceutical combination comprising: (i) a first pharmaceutical composition comprising in a unit dosage form an N-methyl-D-aspartate (NMDA) receptor antagonist or a pharmaceutically acceptable salt thereof and a pharmaceutically acceptable carrier or excipient; and (ii) a second pharmaceutical composition comprising in a solid unit dosage form S-adenosylmethionine or a pharmaceutically acceptable salt thereof, and a pharmaceutically acceptable carrier or excipient.

In some embodiments, the present disclosure provides a fixed dose combination comprising, in unit dosage form: (i) an N-methyl-D-aspartate (NMDA) receptor antagonist or a pharmaceutically acceptable salt thereof; (ii) S-adenosylmethionine (SAM-e) or a pharmaceutically acceptable salt thereof; and (iii) and a pharmaceutically acceptable carrier or excipient.

### (A) NMDA Receptor Antagonists

An “N-methyl-D-aspartate receptor” (“NMDA receptor”) is an G-protein coupled ionotropic glutamate receptor that plays a role in regulating a wide variety of neurological functions, including breathing, locomotion, learning, memory formation, and neuroplasticity. Structural and functional impairment of the NMDA receptor can lead to neurodegenerative and cognitive disorders, including Alzheimer's disease, Parkinson's disease, Huntington disease, neuropathic pain, epilepsy, and psychiatric disorders. NMDA receptors possess certain unique features that distinguish them from other glutamate receptors, such as α-Amino-3-hydroxy-5-methyl-4-isoxazole propionic acid (AMPA). For example, NMDA receptors require two ligands, glutamate, and glycine, for activation. Moreover, the receptors undergo voltage-dependent block by Mg²⁺ and display high permeability to Ca²⁺ when the Mg²⁺ block is removed. The term NMDA receptor as used herein includes all of the binding site subcategories associated with the NMDA receptor, e.g., the glycine-binding site and the phenylcyclidine (PCP)-binding site.

The complex structure of the NMDA receptor provides multiple sites for potential therapeutic inhibition. A compound that binds to and blocks an NMDA binding site is referred to herein as an “NMDA receptor antagonist”. The NMDA receptor antagonist can be a competitive or non-competitive inhibitor of the NMDA receptor. Competitive NMDA antagonists bind directly to the glutamate site of the NMDA receptor to inhibit the action of glutamate. Non-competitive antagonists block the NMDA-associated ion channel in a use-dependent manner. Other sites on the NMDA receptor susceptible to antagonism are the glycine site and the polyamine site. In some embodiments, the NMDA receptor antagonist is a non-competitive NMDA receptor antagonist (e.g., ketamine or pharmaceutically acceptable salts thereof). In some embodiments, the NMDA receptor antagonist is a competitive NMDA receptor antagonist.

Non-limiting examples of NMDA receptor antagonists that can be used in the compositions and methods of the present disclosure are ketamine (including racemic ketamine, (R)-ketamine (Arketamine) and (S)-ketamine (Esketamine)), dextromethorphan, memantine, amantadine, neramexane, phenylcyclidine, and pharmaceutically acceptable salts, enantiomers, and racemates thereof.

### Ketamine

Ketamine is a non-selective, non-competitive antagonist of the NMDA receptor that can bind to the phenylcyclidine binding site. The chemical name for ketamine is (+)-2-(o-Chlorophenyl)-2-(methylamino)cyclohexanone. Ketamine is represented by the following chemical structure.

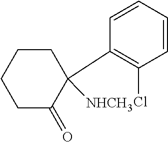 [SMILES: CNC1(c2ccccc2Cl)CCCCC1=O]

In some embodiments, ketamine can be in the form of a pharmaceutically acceptable salt. In some embodiments, ketamine is provided as a hydrochloride (HCl) salt, which is represented by the following structure.

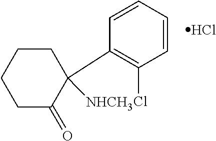 [SMILES: CNC1(c2ccccc2Cl)CCCCC1=O ; Cl]

In some embodiments, the ketamine is racemic ketamine, i.e., it is an equal mixture of the (R) and (S) enantiomer, or pharmaceutically acceptable salts thereof (e.g., HCl salt). In some embodiments, the ketamine is R-ketamine (Arketamine), or a pharmaceutically acceptable salt thereof (e.g., HCl salt). In some embodiments, the ketamine is S-ketamine (Esketamine), or a pharmaceutically acceptable salt thereof (e.g., HCl salt). The structures of R-ketamine and S-ketamine are provided below.

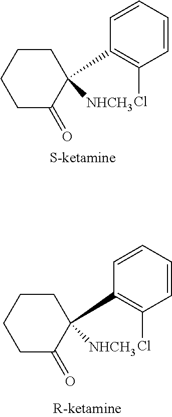 [SMILES: CN[C@]1(c2ccccc2Cl)CCCCC1=O ; CN[C@@]1(c2ccccc2Cl)CCCCC1=O]

In some embodiments, ketamine can be provided as mixtures of its two enantiomers ((R) and (S) ketamine). Mixtures of (R):(S) enantiomers of ketamine can be about 1:99 to about 99:1, for example about 1:99, 5:95, about 10:90, about 15:85, about 20:80, about 25:75, about 30:70, about 35:65, about 40:60, about 45:55, about 50:50 (i.e., racemic ketamine), about 55:45, about 40:60, about 35:65, about 30:70, about 25:75, about 20:80, about 15:85, about 10:80, about 5:95, about 1:99, and any ratio in-between.

Racemic ketamine is indicated for the induction and maintenance of anesthesia (under the brand name Ketalar®). S-ketamine (esketamine), in conjunction with an oral antidepressant, is indicated for the treatment of treatment-resistant depression (TRD) (under the brand name Spravato®). Ketalar® (racemic ketamine HCl injection for intravenous or intramuscular injection), is formulated as a solution for intravenous or intramuscular injection, in 200 mg/20 mL (10 mg/mL), 500 mg/10 mL (50 mg/mL), or 500 mg/5 ml (100 mg/mL) multiple-dose vials (MDVs). Each milliliter (mL) of the multiple-dose vials contain either 10 mg ketamine base (equivalent to 11.53 mg ketamine hydrochloride), 50 mg ketamine base (equivalent to 57.67 mg ketamine hydrochloride) or 100 mg ketamine base (equivalent to 115.33 mg ketamine hydrochloride) and optionally up to about 0.10 mg/mL benzethonium chloride in water for injection. The 10 mg/mL solution can be made isotonic with sodium chloride. Spravato® (Esketamine HCl nasal spray), is contained as a solution in a stoppered glass vial within a nasal spray device. Each device delivers two sprays with a total of 32.3 mg of esketamine hydrochloride (equivalent to 28 mg esketamine) in 0.2 of an aqueous solution with a pH of 4.5. Inactive ingredients include citric acid monohydrate, edetate disodium, sodium hydroxide, and water for injection.

Ketamine or a pharmaceutically acceptable salt thereof can be administered to a human patient in a variety of forms adapted to the chosen route of administration, e.g., oral, parenteral, (e.g., intravenous, intramuscular, subcutaneous), intraocular, intrasynovial, transepithelial (including transdermal, ophthalmic, sublingual and buccal); topical (e.g., ophthalmic, dermal, ocular), rectal and nasal, as described herein. In some embodiments, a commercial formulation of ketamine can be used in the combinations and methods of the present disclosure.

### Dextromethorphan

Dextromethorphan is a noncompetitive NMDA receptor antagonist and sigma-1 receptor agonist. The chemical name of dextromethorphan is 3-methoxy-17-methyl-(9a, 13a, 14a)-morphinan. In some embodiments, dextromethorphan can be in the form of a pharmaceutically acceptable salt. In some embodiments, dextromethorphan is provided as a hydrobromide (HBr) salt. In some embodiments, dextromethorphan is provided as dextromethorphan hydrobromide monohydrate, which is represented by the following structure.

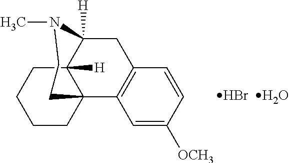 [SMILES: COc1ccc2c(c1)[C@]13CCCC[C@@H]1[C@H](C2)N(C)CC3]

Dextromethorphan can be formulated as a suspension comprising dextromethorphan or a pharmaceutically acceptable salt thereof (e.g., dextromethorphan HBr) and inactive ingredients such as citric acid, disodium edetate, ethylcellulose, high fructose corn syrup, methylparaben, partially hydrogenated vegetable oil (soybean, cottonseed), polyethylene glycol 3350, polysorbate 80, propylene glycol, propylparaben, water, sucrose, tragacanth, xanthan gum, and colorings.

Dextromethorphan can also be formulated as a tablet (immediate or extended release) for oral administration, comprising dextromethorphan or a pharmaceutically acceptable salt thereof (e.g., Dextromethorphan HBr) and inactive ingredients such as carbomer homopolymer, colloidal silicon dioxide, crospovidone, glyceryl monocaprylocaprate, I-cysteine hydrochloride monohydrate, magnesium stearate, microcrystalline cellulose, polyvinyl alcohol, red iron oxide, sodium lauryl sulfate, stearic acid, talc, titanium dioxide, and yellow iron oxide.

Dextromethorphan or a pharmaceutically acceptable salt thereof can be administered to a human patient in a variety of forms adapted to the chosen route of administration, e.g., oral, parenteral, (e.g., intravenous, intramuscular, subcutaneous), intraocular, intrasynovial, transepithelial (including transdermal, ophthalmic, sublingual and buccal); topical (e.g., ophthalmic, dermal, ocular), rectal and nasal, as described herein. In some embodiments, a commercial formulation of dextromethorphan can be used in the combinations and methods of the present disclosure.

### Memantine

Memantine (sold under the brand name Namenda®) is an oral NMDA receptor antagonist. The chemical name for memantine is 1-amino-3,5-dimethyladamantane. In some embodiments, memantine can be in the form of a pharmaceutically acceptable salt. In some embodiments, memantine is provided as a hydrochloride (HCl) salt, which is represented by the following structure.

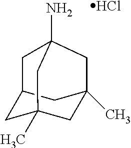

Memantine is available for oral administration as capsule-shaped, film-coated tablets containing 5 mg and 10 mg of memantine hydrochloride. The tablets also contain the following inactive ingredients: microcrystalline cellulose/colloidal silicon dioxide, talc, croscarmellose sodium, and magnesium stearate. The film coat contains hypromellose, titanium dioxide, polyethylene glycol 400, and colorants. Alternatively, memantine is available for oral administration as 7, 14, 21 and 28 mg capsules. Each capsule contains extended release beads with the labeled amount of memantine HCl and the following inactive ingredients: sugar spheres, polyvinylpyrrolidone, hypromellose, talc, polyethylene glycol, ethylcellulose, ammonium hydroxide, oleic acid, and medium chain triglycerides in hard gelatin capsules.

Memantine or a pharmaceutically acceptable salt thereof may be administered to a human patient in a variety of forms adapted to the chosen route of administration, e.g., oral, parenteral, (e.g., intravenous, intramuscular, subcutaneous), intraocular, intrasynovial, transepithelial (including transdermal, ophthalmic, sublingual and buccal); topical (e.g., ophthalmic, dermal, ocular), rectal and nasal, as described herein. In some embodiments, a commercial formulation of memantine can be used in the combinations and methods of the present disclosure.

### Amantadine

Amantadine is an uncompetitive NMDA receptor antagonist and sigma-1 receptor agonist. The chemical name of amantadine is 1-adamantanamine, and its chemical structure is shown below.

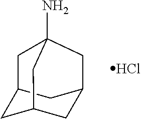 [SMILES: NC12CC3CC(CC(C3)C1)C2 ; Cl]

Amantadine is available as extended release capsules for oral administration. Each capsule contains 68.5 mg or 137 mg amantadine (as 85 mg or 170 mg amantadine hydrochloride, respectively). Capsules also contain the following inactive ingredients: copovidone, ethylcellulose, hypromellose, magnesium stearate, medium-chain triglycerides, microcrystalline cellulose, povidone, and talc in a hard gelatin capsule.

Amantadine or a pharmaceutically acceptable salt thereof may be administered to a human patient in a variety of forms adapted to the chosen route of administration, e.g., oral, parenteral, (e.g., intravenous, intramuscular, subcutaneous), intraocular, intrasynovial, transepithelial (including transdermal, ophthalmic, sublingual and buccal); topical (e.g., ophthalmic, dermal, ocular), rectal and nasal, as described herein. In some embodiments, a commercial formulation of amantadine can be used in the combinations and methods of the present disclosure.

### Neramexane

Neramexane is a drug related to memantine, which acts as an NMDA antagonist and has neuroprotective effects. The chemical name of neramexane is 1,3,3,5,5-pentamethylcyclohexanamine, and its chemical structure is shown below.

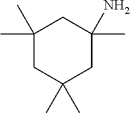

Neramexane or a pharmaceutically acceptable salt thereof may be administered to a human patient in a variety of forms adapted to the chosen route of administration, e.g., oral, parenteral, (e.g., intravenous, intramuscular, subcutaneous), intraocular, intrasynovial, transepithelial (including transdermal, ophthalmic, sublingual and buccal); topical (e.g., ophthalmic, dermal, ocular), rectal and nasal, as described herein.

### Phenylcyclidine

Phencyclidine or phenylcyclohexyl piperidine (PCP), is an NMDA antagonist. In addition to its interactions with NMDA receptors, PCP has also been shown to inhibit dopamine reuptake, and thereby leads to increased extracellular levels of dopamine and hence increased dopaminergic neurotransmission. The chemical structure of PCP is shown below.

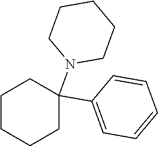 [SMILES: c1ccc(C2(N3CCCCC3)CCCCC2)cc1]

Phencyclidine or a pharmaceutically acceptable salt thereof may be administered to a human patient in a variety of forms adapted to the chosen route of administration, e.g., oral, parenteral, (e.g., intravenous, intramuscular, subcutaneous), intraocular, intrasynovial, transepithelial (including transdermal, ophthalmic, sublingual and buccal); topical (e.g., ophthalmic, dermal, ocular), rectal and nasal, as described herein. In some embodiments, a commercial formulation of PCP can be used in the combinations and methods of the present disclosure.

### (B) S-adenosylmethionine (SAM-e)

S-adenosylmethionine (SAM-e) is an endogenous molecule that plays an important role in cellular metabolism. The structure of SAM-e is shown below.

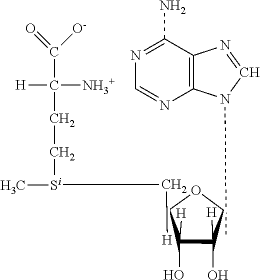

SAM-e is formed through combination of the essential amino acid methionine with adenosine triphosphate (ATP). The reactions that produce, consume, and regenerate SAM are called the SAM cycle. In the first step of this cycle, the SAM-dependent methylases that use SAM as a substrate produce S-adenosyl homocysteine as a product. This is hydrolyzed to homocysteine and adenosine. The homocysteine is recycled back to methionine. This methionine can then be converted back to SAM, completing the cycle.

SAM-e is required for the synthesis of monoamine neurotransmitters, such as norepinephrine (NE), dopamine (DA) and serotonin [5-hydroxytryptophan (5-HT)], which play important roles in maintaining normal mood. SAM-e is used as a dietary supplement in capsules or tablets, in the United States, and is approved as a prescription drug for depression in Europe.

In some embodiments, SAM-e can be provided in the form of a pharmaceutically acceptable salt. In some embodiments, the pharmaceutically acceptable salt is S-adenosylmethionine disulfate tosylate. In some embodiments, the pharmaceutically acceptable salt is S-adenosylmethionine 1,4-butanedisulfonate. In some embodiments, the pharmaceutically acceptable salt is S-adenosylmethionine phytate.

SAM-e or a pharmaceutically acceptable salt thereof may be administered to a human patient in a variety of forms adapted to the chosen route of administration, e.g., oral, parenteral, (e.g., intravenous, intramuscular, subcutaneous), intraocular, intrasynovial, transepithelial (including transdermal, ophthalmic, sublingual and buccal); topical (e.g., ophthalmic, dermal, ocular), rectal and nasal, as described herein. In some embodiments, a commercial formulation (e.g., capsule or tablet) can be used in the combinations and methods of the present disclosure.

### Pharmaceutically Acceptable SALTS

The disclosure provides the use of pharmaceutically acceptable salts of any therapeutic compound described herein. Pharmaceutically acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically acceptable salt is a metal salt. In some embodiments, a pharmaceutically acceptable salt is an ammonium salt.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc. In some embodiments, the salt is an ammonium salt.

Acid addition salts can arise from the addition of an acid to a compound of the present disclosure. In some embodiments, the acid is organic. In some embodiments, the acid is inorganic. In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a trifluoroacetate salt, a mandelate salt, a cinnamate salt, an aspartate salt, a stearate salt, a palmitate salt, a glycolate salt, a propionate salt, a butyrate salt, a fumarate salt, a hemifumarate salt, a succinate salt, a methanesulfonate salt, an ethanesulfonate salt, a benzenesulfonate salt, a p-toluenesulfonate salt, a citrate salt, an oxalate salt, a phytate salt, or a maleate salt.

In some embodiments, the combinations of the present disclosure comprise an NMDA receptor antagonist in the form of a pharmaceutically acceptable salt. In some embodiments, the combinations of the present disclosure comprise ketamine in the form of a pharmaceutically acceptable salt. In some embodiments, the salt is a hydrochloride salt.

In some embodiments, the combinations of the present disclosure comprise S-adenosylmethionine in the form of a pharmaceutically acceptable salt. In some embodiments, the salt is a p-toluenesulfonate (tosylate) salt. In some embodiments, the salt is a disulfide tosylate salt of S-adenosylmethionine. In some embodiments, the salt is a phytate salt of S-adenosylmethionine.

### Combinations of NMDA Receptor Antagonists and SAM-e

In some embodiments, the present disclosure provides a combination of an N-methyl-D-aspartate (NMDA) receptor antagonist or a pharmaceutically acceptable salt thereof and S-adenosylmethionine (SAM-e) or a pharmaceutically acceptable salt thereof. In some embodiments, the present disclosure provides a method of treating a neurological disorder by administering a combination of an N-methyl-D-aspartate (NMDA) receptor antagonist or a pharmaceutically acceptable salt thereof and S-adenosylmethionine (SAM-e) or a pharmaceutically acceptable salt thereof.

In some embodiments, the combination of the present disclosure provides an additive therapeutic effect, i.e., the combined effect of the NMDA receptor antagonist and the SAM-e equals the sum of the effects of the two drugs acting independently. In some embodiments, an additive effect can comprise a therapeutic response that is the same or similar to the therapeutic effect of either of the NMDA receptor or SAM-e, but the adverse effects associated with either the NMDA receptor or SAM-e is reduced as compared with the therapeutic effect of that drug administered in monotherapy.

In some embodiments, the combination of the present disclosure provides a synergistic therapeutic effect, i.e., the combined effect of the NMDA receptor antagonist and the SAM-e is greater than the sum of the effects of the two drugs acting independently.

Ketamine's mechanism of action is thought to be related to the presynaptic inhibition of glutamate release. Ketamine decreases neuronally released glutamate via retrograde stimulation of presynaptic adenosine A1 receptors (A1R). Specifically, ketamine or a ketamine metabolite acts on α-amino-3-hydroxy-5-methyl-4-isoxazole propionic acid receptor (AMPA receptor, AMPAR), an ionotropic transmembrane receptor for glutamate (iGluR) that mediates fast synaptic transmission in the central nervous system. Ketamine, acting on AMPARS, may evoke a postsynaptic adenosine release, which retrogradely feedbacks onto A1R located on glutamatergic terminals and induce presynaptic inhibition of synaptic recycling and glutamate release. As mentioned above, the SAM cycle produces adenosine as a hydrolysis product of S-adenosylhomocysteine. It is contemplated that the additional adenosine from SAM-e metabolism can enhance the therapeutic effect of ketamine.

In some embodiments, combining an NMDA-receptor antagonist such as ketamine or a pharmaceutically acceptable salt thereof and SAM-e or a pharmaceutically acceptable salt thereof can result in reduced side effects associated with ketamine monotherapy, for example reduced hypertension, hepatotoxicity and/or reduced dissociation experiences.

In an embodiment, combining an NMDA-receptor antagonist such as ketamine or a pharmaceutically acceptable salt thereof and SAM-e or a pharmaceutically acceptable salt thereof can result in reduced hypertensive effects. For example, an NMDA-receptor antagonist such as ketamine typically causes an increase in blood pressure, requiring continuous monitoring during the infusion. Up to 30% of patients need to be treated during an infusion with an antihypertensive medication. SAM-e can cause hypotension that may counteract ketamine-induced hypertension. Thus, in some embodiments, the present disclosure provides a method of reducing hypertension in a subject receiving an N-methyl-D-aspartate (NMDA) receptor antagonist or a pharmaceutically acceptable salt thereof to treat a neurological condition, the method comprising administering to the subject a combination comprising a therapeutically effective amount of the NMDA receptor antagonist or pharmaceutically acceptable salt thereof, and a therapeutically effective amount of S-adenosylmethionine (SAM-e) or a pharmaceutically acceptable salt thereof. In some embodiments, the present disclosure provides a method of reducing hypertension in a subject receiving ketamine or a pharmaceutically acceptable salt thereof to treat a neurological condition, the method comprising administering to the subject a combination comprising a therapeutically effective amount of the ketamine or pharmaceutically acceptable salt thereof, and a therapeutically effective amount of S-adenosylmethionine (SAM-e) or a pharmaceutically acceptable salt thereof. In an embodiment, combining an NMDA-receptor antagonist such as ketamine or a pharmaceutically acceptable salt thereof and SAM-e or a pharmaceutically acceptable salt thereof can result in reduced hepatotoxicity. For example, an NMDA-receptor antagonist such as ketamine can cause hepatobiliary dysfunction and/or drug-induced liver injury (DILI). SAM-e can improve liver function (e.g., decrease transaminase levels) or liver disease outcomes in hepatitis, alcoholic and viral liver cirrhosis and cholestasis, and has also been shown to improve biochemical liver parameters and symptoms of cholestasis. Therefore, SAM-e may counteract ketamine-induced hepatotoxicity. Thus, in some embodiments, the present disclosure provides a method of reducing hepatotoxicity in a subject receiving an N-methyl-D-aspartate (NMDA) receptor antagonist or a pharmaceutically acceptable salt thereof to treat a neurological condition, the method comprising administering to the subject a combination comprising a therapeutically effective amount of the NMDA receptor antagonist or pharmaceutically acceptable salt thereof, and a therapeutically effective amount of S-adenosylmethionine (SAM-e) or a pharmaceutically acceptable salt thereof. In some embodiments, the present disclosure provides a method of reducing hepatotoxicity in a subject receiving ketamine or a pharmaceutically acceptable salt thereof to treat a neurological condition, the method comprising administering to the subject a combination comprising a therapeutically effective amount of the ketamine or pharmaceutically acceptable salt thereof, and a therapeutically effective amount of S-adenosylmethionine (SAM-e) or a pharmaceutically acceptable salt thereof.

In an embodiment, combining an NMDA-receptor antagonist such as ketamine or a pharmaceutically acceptable salt thereof and SAM-e or a pharmaceutically acceptable salt thereof can result in reduced dissociation effects. Dissociation refers to a temporary mental state in which a person ‘detaches’ from their surroundings, i.e., the person becomes less aware of what is actually around them and starts to feel disconnected from their body. Individuals can report altered consciousness and perceptions of themselves and their environment. NMDA receptor antagonists, in particular non-competitive NMDA receptor antagonists, can cause dose-dependent dissociation. The product label for Esketamine states that patients taking the drug are at risk for dissociative or perceptual changes after administration. Because of the risks of sedation and dissociation, patients must be monitored for at least 2 hours at each treatment session, followed by an assessment to determine when the patient is considered clinically stable and ready to leave the healthcare setting. This places a burden on the patient as well as the healthcare system. Furthermore, intolerance of dissociation may make a patient unable to receive treatment. In some embodiments, adenosine, a metabolite of SAM-e, can reduce dissociation that is linked to ketamine treatment. The SAM cycle releases adenosine, which acts as a neuromodulator, primarily via pre- and postsynaptic adenosine A1 (A1R) and A2 (A2R) receptor subtypes. With this modulatory role on neurotransmission, adenosine can inhibit excitatory neurons without interfering directly with postsynaptic GABA or glutamate receptors. Strong neuronal activity (which can occur during ketamine infusion) in the nervous system can lead to elevated extracellular levels of adenosine. At the same time, A1Rs inhibit synaptic transmission; thus, adenosine is an inhibitory neuromodulator and therefore can be useful in conditions involving neural overactivity such as ketamine-induced dissociation.

Thus, in some embodiments, the present disclosure provides a method of reducing a dissociative effect in a subject receiving an N-methyl-D-aspartate (NMDA) receptor antagonist or a pharmaceutically acceptable salt thereof to treat a neurological condition, the method comprising administering to the subject a combination comprising a therapeutically effective amount of the NMDA receptor antagonist or pharmaceutically acceptable salt thereof, and a therapeutically effective amount of S-adenosylmethionine (SAM-e) or a pharmaceutically acceptable salt thereof. In some embodiments, the present disclosure provides a method of reducing a dissociative effect in a subject receiving ketamine or a pharmaceutically acceptable salt thereof to treat a neurological condition, the method comprising administering to the subject a combination comprising a therapeutically effective amount of the ketamine or pharmaceutically acceptable salt thereof, and a therapeutically effective amount of S-adenosylmethionine (SAM-e) or a pharmaceutically acceptable salt thereof.

### Therapeutic Methods

As contemplated herein, the present disclosure provides a method of treating a neurological condition in a subject in need thereof, the method comprising administering to the subject a combination comprising a therapeutically effective amount of an N-methyl-D-aspartate (NMDA) receptor antagonist (e.g., ketamine) or a pharmaceutically acceptable salt thereof, and a therapeutically effective amount of S-adenosylmethionine or a pharmaceutically acceptable salt thereof.

Examples of neurological disorders that may be treated, or that may be treated by the combinations of the present disclosure include, but are not limited to affective disorders, psychiatric disorders, cerebral function disorders, movement disorders, dementias, motor neuron diseases, neurodegenerative diseases, seizure disorders, and headaches.

In some embodiments, the neurological condition is Major Depressive Disorder (MDD). In some embodiments, the neurological condition is Treatment Resistant Depression (TRD). In some embodiments, the neurological condition is Suicidal Ideation (SI). In some embodiments, the neurological condition is Substance Use Disorder (SUD). In some embodiments, the neurological condition is Complex Regional Pain Syndrome (CRPS). In some embodiments, the neurological condition is fibromyalgia (FM). In some embodiments, the neurological condition is Alzheimer's Disease (AD). In some embodiments, the neurological condition is Attention Deficit Disorder (ADD). In some embodiments, the neurological condition is Attention Deficit Hyperactive Disorder (ADHD). In some embodiments, the neurological condition is obesity. In some embodiments, the neurological condition is Post Traumatic Stress Disorder (PTSD). In some embodiments, the neurological condition is Generalized Anxiety Disorder (GAD).

The term “treatment-resistant depression” (TRD), also known as treatment-refractory depression, is a condition generally associated with patients who have failed treatment with at least two antidepressants from different classes. The diagnosis of TRD requires a patient to have had an inadequate response to treatment with the antidepressants after an adequate dose and adequate course, e.g. during the current depressive episode. TRD may be more difficult to treat due to the comorbidity of other medical or psychological illnesses, such as drug/alcohol abuse or eating disorders, or TRD being misdiagnosed. Some TRD patients have had an inadequate response to 1, 2, 3, or more adequate antidepressant treatment or have failed or had an inadequate response to 1, 2, 3, or more prior antidepressant treatments.

Affective disorders that may be treated by the combinations of the present disclosure include, but are not limited to, depression, major depression, treatment resistant depression and treatment resistant bipolar depression, bipolar disorders including cyclothymia, seasonal affective disorder, mood disorders, chronic depression (dysthymia), psychotic depression, postpartum depression, premenstrual dysphoric disorder (PMDD), situational depression, atypical depression, anxiety disorders, attention deficit disorder (ADD), attention deficit disorder with hyperactivity (ADDH), and attention deficit/hyperactivity disorder (AD/HD), bipolar and manic conditions, obsessive-compulsive disorder, anorexia nervosa, bulimia, obesity or weight-gain, narcolepsy, chronic fatigue syndrome, premenstrual syndrome, substance addiction or abuse, nicotine addiction, psycho-sexual dysfunction, pseudobulbar affect, and emotional lability.

Depression may be manifested by depressive symptoms. These symptoms may include psychological changes such as changes in mood, feeling sad, despair, mental slowing, loss of concentration, pessimistic thoughts, agitation, anxiety, irritability, guilt, anger, feelings of worthlessness, reckless behavior, suicidal thoughts or attempts, and/or self-deprecation. Physical symptoms of depression may include insomnia, anorexia, appetite loss, weight loss, weight gain, decreased energy and libido, fatigue, restlessness, aches, pains, headaches, cramps, digestive issues, and/or abnormal hormonal circadian rhythms. Any one or more of these symptoms can be treated with the combinations of the present disclosure.

Psychiatric disorders that may be treated by combinations of the present disclosure include, but are not limited to, anxiety disorders, including but not limited to, phobias, generalized anxiety disorder, social anxiety disorder, panic disorder, agoraphobia, obsessive-compulsive disorder, and post-traumatic stress disorder (PTSD); bipolar depression, hypomania, unipolar depression, depression, stress disorders, somatoform disorders, personality disorders, psychosis, schizophrenia, delusional disorder, schizoaffective disorder, schizotypy, aggression, aggression in Alzheimer's disease, agitation, and agitation in Alzheimer's disease.

Alzheimer's disease (AD) is a progressive neurodegenerative disorder characterized by cognitive decline, and behavioral and psychological symptoms including agitation. AD is the most common form of dementia and afflicts millions of individuals in the United States. Agitation in Alzheimer's disease occurs as the disease progresses. Agitation is reported in up to 70% of patients with AD and is characterized by emotional distress, aggressive behaviors, disruptive irritability, and disinhibition. Managing agitation is a priority in AD. Agitation in patients with AD has been associated with increased caregiver burden, decreased functioning, accelerated cognitive decline, earlier nursing home placement, and increased mortality. There are currently no therapies approved by the FDA for the treatment of agitation in patients with AD.

Agitation in Alzheimer's disease and other neurological disorders may present itself as inappropriate verbal, emotional, and/or physical behaviors. Inappropriate behaviors may include, but are not limited to, incoherent babbling, inappropriate emotional response, demands for attention, threats, irritability, frustration, screaming, repetitive questions, mood swings, cursing, abusive language, physical outbursts, emotional distress, restlessness, shredding, sleeping disturbances, delusions, hallucinations, pacing, wandering, searching, rummaging, repetitive body motions, hoarding, shadowing, hitting, scratching, biting, combativeness, hyperactivity, and/or kicking. In some embodiments, treatment of agitation in Alzheimer's disease may result in a reduction of agitation-related symptoms of at least about 5%, at least about 10%, at least about 20%, at least about 30%, at least about 40%, at least about 50%, at least about 60%, at least about 70%, at least about 80%, at least about 90%, up to about 100%, or any other reduction in a range bounded by any of these values.

Cerebral function disorders that may be treated by the combinations of the present disclosure include, but are not limited to, disorders involving intellectual deficits such as senile dementia, Alzheimer's type dementia, memory loss, amnesia/amnestic syndrome, epilepsy, disturbances of consciousness, coma, lowering of attention, speech disorders, voice spasms, Parkinson's disease, Lennox-Gastaut syndrome, autism, hyperkinetic syndrome, and schizophrenia. Cerebral function disorders also include disorders caused by cerebrovascular diseases including, but not limited to, stroke, cerebral infarction, cerebral bleeding, cerebral arteriosclerosis, cerebral venous thrombosis, head injuries, and the like where symptoms include disturbance of consciousness, senile dementia, coma, lowering of attention, and speech disorders.

Movement disorders that may be treated by the combinations of the present disclosure include, but are not limited to, akathisia, akinesia, associated movements, athetosis, ataxia, ballismus, hemiballismus, bradykinesia, cerebral palsy, chorea, Huntington's disease, rheumatic chorea, Sydenham's chorea, dyskinesia, tardive dyskinesia, dystonia, blepharospasm, spasmodic torticollis, dopamine-responsive dystonia, Parkinson's disease, restless legs syndrome (RLS), tremor, essential tremor, and Tourette's syndrome, and Wilson's disease.

Dementias that may be treated by the combinations of the present disclosure include, but are not limited to, Alzheimer's disease, Parkinson's disease, vascular dementia, dementia with Lewy bodies, mixed dementia, fronto-temporal dementia, Creutzfeldt-Jakob disease, normal pressure hydrocephalus, Huntington's disease, Wernicke-Korsakoff Syndrome, and Pick's disease.

Motor neuron diseases that may be treated by the combinations of the present disclosure include, but are not limited to, amyotrophic lateral sclerosis (ALS), progressive bulbar palsy, primary lateral sclerosis (PLS), progressive muscular atrophy, post-polio syndrome (PPS), spinal muscular atrophy (SMA), spinal motor atrophies, Tay-Sach's disease, Sandoff disease, and hereditary spastic paraplegia.

Neurodegenerative diseases that may be treated by the combinations of the present disclosure include, but are not limited to, Alzheimer's disease, prion-related diseases, cerebellar ataxia, spinocerebellar ataxia (SCA), spinal muscular atrophy (SMA), bulbar muscular atrophy, Friedrich's ataxia, Huntington's disease, Lewy body disease, Parkinson's disease, amyotrophic lateral sclerosis (ALS or Lou Gehrig's disease), multiple sclerosis (MS), multiple system atrophy, Shy-Drager syndrome, corticobasal degeneration, progressive supranuclear palsy, Wilson's disease, Menkes disease, adrenoleukodystrophy, cerebral autosomal dominant arteriopathy with subcortical infarcts and leukoencephalopathy (CADASIL), muscular dystrophies, Charcot-Marie-Tooth disease (CMT), familial spastic paraparesis, neurofibromatosis, olivopontine cerebellar atrophy or degeneration, striatonigral degeneration, Guillain-Barre syndrome, and spastic paraplesia.

Seizure disorders that may be treated by the combinations of the present disclosure include, but are not limited to, epileptic seizures, nonepileptic seizures, epilepsy, febrile seizures; partial seizures including, but not limited to, simple partial seizures, Jacksonian seizures, complex partial seizures, and epilepsia partialis continua; generalized seizures including, but not limited to, generalized tonic-clonic seizures, absence seizures, atonic seizures, myoclonic seizures, juvenile myoclonic seizures, and infantile spasms; and status epilepticus.

Other neurological disorders that may be treated by the combinations of the present disclosure include Rett Syndrome, autism, tinnitus, disturbances of consciousness disorders, sexual dysfunction, intractable coughing, narcolepsy, cataplexy; voice disorders due to uncontrolled laryngeal muscle spasms, including, but not limited to, abductor spasmodic dysphonia, adductor spasmodic dysphonia, muscular tension dysphonia, and vocal tremor; diabetic neuropathy, chemotherapy-induced neurotoxicity, such as methotrexate neurotoxicity; incontinence including, but not limited, stress urinary incontinence, urge urinary incontinence, and fecal incontinence; and erectile dysfunction.

In some embodiments, treatment of a neurological condition with an N-methyl-D-aspartate (NMDA) receptor antagonist or pharmaceutically acceptable salt thereof can be associated with a side effect. In some embodiments, treatment of a neurological condition with an NMDA receptor antagonist or pharmaceutically acceptable salt thereof can be associated with hypertension. In some embodiments, administering the NMDA receptor antagonist or pharmaceutically acceptable salt thereof to a subject in combination with S-adenosylmethionine can result in reduced hypertension in the subject as compared with hypertension measured in the subject after receiving the NMDA receptor antagonist or pharmaceutically acceptable salt thereof as monotherapy.

In some embodiments, treatment of a neurological condition with an NMDA receptor antagonist or pharmaceutically acceptable salt thereof can be associated with hepatotoxicity. In some embodiments, administering the NMDA receptor antagonist or pharmaceutically acceptable salt thereof to a subject in combination with S-adenosylmethionine can result in reduced hepatotoxicity in the subject as compared with hypertension measured in the subject after receiving the NMDA receptor antagonist or pharmaceutically acceptable salt thereof as monotherapy.

In some embodiments, treatment of a neurological condition with an NMDA receptor antagonist or pharmaceutically acceptable salt thereof can be associated with dissociative experiences. In some embodiments, administering the NMDA receptor antagonist or pharmaceutically acceptable salt thereof to a subject in combination with S-adenosylmethionine can result in reduced dissociation in the subject as compared with hypertension measured in the subject after receiving the NMDA receptor antagonist or pharmaceutically acceptable salt thereof as monotherapy.

### Measure of Therapeutic Effect

Measures of treatment effect that may be improved by treatment with a combination of an NMDA receptor antagonist (e.g., ketamine) and S-adenosylmethionine (SAM-e) include but are not limited to, Neuropsychiatric Inventory-Clinician (NPI-C) rating scale, overall and all domains; Neuropsychiatric Inventory-Clinician (NPI-C) rating scale Agitation domain; Cohen-Mansfield Agitation Inventory (CMAI); Cornell Scale for Depression in Dementia (CSDD); Neuropsychiatric Inventory (NPI Agitation/Aggression Domain); Cocomitant Medications (Frequency of using concomitant medications); Alzheimer's Disease Cooperative Study-Activities of Daily Living Inventory (ADCS-ADL); Neuropsychiatric Inventory (NPI) Individual Domains and NPI Total Scores (range 0-144), including NPI-C Apathy domain, NPI Agitation/Aggression Caregiver Distress, Modified Alzheimer's Disease Cooperative Study-Clinical Global Impression of Change Agitation (mADCS-CGIC Agitation), Patient Global Impression of Change (PGIC) (rated by caregiver), Dementia Quality of Life (DEMQOL), Quality of Life-Alzheimer's disease measure (QoL-AD), Zarit Burden Scale, Resource Utilization in Dementia (RUD), Alzheimer's Disease Assessment Scale-Cognitive Subscale (ADAS-Cog), Mini-mental State Examination (MMSE), Caregiver Strain Index (CSI), Individual Domain of the Neuropsychiatric Inventory (NPI), Total Neuropsychiatric Inventory (NPI) Score, Neuropsychiatric Inventory (Agitation/Aggression Domain of NPI), Neuropsychiatric Inventory (Caregiver Distress for NPI Domains), and the like.

In some embodiments, treating a person with a combination of an NMDA receptor antagonist (e.g., Ketamine) and S-adenosylmethionine (SAM-e) may improve (e.g. reduce) the person's score in one of the above assessments by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 90%, at least about 95%, about 100%, about 10-20%, about 20-30%, about 30-40%, about 40-50%, about 5-15%, about 15-25%, about 25-35%, about 35-45%, about 45-55%, about 50-60%, about 60-70%, about 70-80%, about 80-90%, about 90-100% as compared to baseline (i.e., before intervention), or as compared to placebo (i.e., no intervention). In some embodiments, the improvement is compared to baseline. In some embodiments, the improvement is compared to placebo.

Administering a combination according to the present disclosure may result in a rapid treatment effect, e.g. within about 1 day, about 2 days, about 3 days, about 4 days, about 5 days, about 6 days, about 1 week, within about 2 weeks, within about 3 weeks, or within about 4 weeks of beginning the treatment.

Patients treated with any of the foregoing described embodiments with the combinations of the present disclosure can be assessed for an improvement using any of the assessments described herein, including, but not limited to MADRS, Quality of Life Enjoyment and Satisfaction Questionnaire Short Form, Range of Impaired Functioning Tool, PRISE, C-SSRS, QIDS-SR), CGI, CPFQ, HAM-D17, MGH ATRQ, CGI-S, CGI-C, EQ-5D-5L, PGIC, GAD-7, CGI-I, SDS, QIDS-SR16, HAM-A, CPFQ, CPFQ-Cognitive subscales (Items 4 to 7), BPRS, DSST, RAVLT, TMT, STROOP, SRT, CRT, CHRT, MDI, etc., which can be observed by treatment of a patient in need.

### Subjects

The pharmaceutical combinations disclosed herein are suitable for administration to human or animal subjects in a biologically compatible form suitable for administration in vivo. Subjects can be, for example, elderly adults, adults, pediatric (adolescents, pre-adolescents, children, toddlers, infants, neonates), and non-human animals. In some embodiments, a subject is a patient. Pediatric patients include patients under about 18 years of age, for example about 0-5 years of age, about 5-10 years of age, about 10-12 years of age, or about 12-18 years of age. Adult patients include patients having an age of 18 years or older, for example about 18-70 years, about 18-65 years, about 18-30 years, about 10-20 years, about 20-30 years, about 30-40 years, about 40-50 years, about 50-60 years, about 60-70 years, about 70-80 years, about 80-90 years, about 30-50 years, about 50-65 years; elderly patients, such as patients over 65 years of age, about 65-75 years of age, about 75-90 years of age, or over 90 years of age.

### Dosing and Dosing Schedules

Compounds according to the present disclosure may be administered to a human patient in a variety of forms adapted to the chosen route of administration, e.g., orally or parenterally. Parenteral administration includes administration by the following routes: intravenous, intramuscular, subcutaneous, intraocular, intrasynovial, transepithelial including transdermal, ophthalmic, sublingual and buccal; topical including ophthalmic, dermal, ocular, rectal, and nasal inhalation via insufflation. In some embodiments, a compound can be formulated for parenteral, intravenous, intramuscular, or subcutaneous administration.

The active ingredients in the combination of the present disclosure can be formulated in separate pharmaceutical compositions, or they can be contained in a single pharmaceutical composition as a fixed dose combination (FDC). When formulated in separate pharmaceutical compositions, each active ingredient can be formulated for a chosen route of administration independently of the other active ingredient.

In some embodiments, the NMDA receptor antagonist (e.g., ketamine) or pharmaceutically acceptable salt thereof in the combinations of the present disclosure is administered parenterally, intravenously, intramuscularly, subcutaneously, intranasally, transdermally, topically or orally. The NMDA receptor antagonist (e.g., ketamine) or pharmaceutically acceptable salt thereof can also be administered sublingually. The NMDA receptor antagonist (e.g., ketamine) or pharmaceutically acceptable salt thereof can also be administered buccally. In some embodiments, the NMDA receptor antagonist (e.g., ketamine) or pharmaceutically acceptable salt thereof is administered parenterally. In some embodiments, the NMDA receptor antagonist (e.g., ketamine) or pharmaceutically acceptable salt thereof is administered intravenously. In some embodiments, the NMDA receptor antagonist (e.g., ketamine) or pharmaceutically acceptable salt thereof is administered intramuscularly. In some embodiments, the NMDA receptor antagonist (e.g., ketamine) or pharmaceutically acceptable salt thereof is administered subcutaneously. In some embodiments, the NMDA receptor antagonist (e.g., ketamine) or pharmaceutically acceptable salt thereof is administered intranasally. In some embodiments, the NMDA receptor antagonist (e.g., ketamine) or pharmaceutically acceptable salt thereof is administered orally. In some embodiments, the NMDA receptor antagonist (e.g., ketamine) or pharmaceutically acceptable salt thereof is administered topically. In some embodiments, the NMDA receptor antagonist (e.g., ketamine) or pharmaceutically acceptable salt thereof is administered rectally. In some embodiments, the S-adenosylmethionine (SAM-e) or pharmaceutically acceptable salt thereof in the combinations of the present disclosure is administered parenterally, intravenously, intramuscularly, subcutaneously, intranasally, transdermally, topically or orally. SAM-e or pharmaceutically acceptable salt thereof can also be administered sublingually. SAM-e or pharmaceutically acceptable salt thereof can also be administered buccally. In some embodiments, SAM-e or a pharmaceutically acceptable salt thereof is administered parenterally. In some embodiments, SAM-e or a pharmaceutically acceptable salt thereof is administered intravenously. In some embodiments, SAM-e or a pharmaceutically acceptable salt thereof is administered intramuscularly. In some embodiments, SAM-e or a pharmaceutically acceptable salt thereof is administered subcutaneously. In some embodiments, SAM-e or a pharmaceutically acceptable salt thereof is administered intranasally. In some embodiments, SAM-e or a pharmaceutically acceptable salt thereof is administered orally. In some embodiments, SAM-e or a pharmaceutically acceptable salt thereof is administered topically. In some embodiments, SAM-e or a pharmaceutically acceptable salt thereof is administered rectally.

In some embodiments, the NMDA receptor antagonist (e.g., ketamine) or pharmaceutically acceptable salt thereof and the S-adenosylmethionine or pharmaceutically acceptable salt thereof are each administered parenterally. In some embodiments, the NMDA receptor antagonist (e.g., ketamine) or pharmaceutically acceptable salt thereof and the S-adenosylmethionine or pharmaceutically acceptable salt thereof are each administered orally. In some embodiments, the NMDA receptor antagonist (e.g., ketamine) or pharmaceutically acceptable salt thereof is administered parenterally, and the S-adenosylmethionine or pharmaceutically acceptable salt thereof is administered orally. In some embodiments, the NMDA receptor antagonist (e.g., ketamine) or pharmaceutically acceptable salt thereof is administered orally, and the S-adenosylmethionine or pharmaceutically acceptable salt thereof is administered parenterally.

In some embodiments, the NMDA receptor antagonist (e.g., ketamine) or pharmaceutically acceptable salt thereof and/or the S-adenosylmethionine or pharmaceutically acceptable salt thereof can be administered once a day, twice a day, thrice a day, four times a day, once every other day, once every 3, 4, 5 or 6 days, once a week, once every two weeks, once a month, or any period in-between. The NMDA receptor antagonist (e.g., ketamine) or pharmaceutically acceptable salt thereof and/or the S-adenosylmethionine or pharmaceutically acceptable salt thereof can be administered for at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about a month, at least about 2 months, about 3 months, about 4 months, about 5 months, about 6 months, about 7 months, about 8 months, about 9 months, about 10 months, about 11 months, or 12 months, or even longer.

In some embodiments, the S-adenosylmethionine or pharmaceutically acceptable salt thereof is administered for a time period (e.g., 1, 2, 3, 4, 5, 6, or 7 days) prior to start of dosing of the NMDA receptor antagonist or a pharmaceutically acceptable salt thereof.

In some embodiments, the NMDA receptor antagonist (e.g., ketamine) or pharmaceutically acceptable salt thereof is administered parenterally (e.g., intravenously or intramuscularly) every other day, and the S-adenosylmethionine or pharmaceutically acceptable salt thereof is administered daily. In some embodiments, the S-adenosylmethionine or pharmaceutically acceptable salt thereof is administered orally one to four times daily. In some embodiments, the S-adenosylmethionine or pharmaceutically acceptable salt thereof is administered orally or parenterally (e.g., intravenously or intramuscularly) on alternating days. For example, on day 1, S-adenosylmethionine or the pharmaceutically acceptable salt thereof is administered orally, on day 2 parenterally, on day 3 orally, on day 4 parenterally, and so forth. Alternatively, on day 1, S-adenosylmethionine or the pharmaceutically acceptable salt thereof is administered parenterally, on day 2 orally, on day 3 parenterally, on day 4 orally, and so forth. In some embodiments, the S-adenosylmethionine or pharmaceutically acceptable salt thereof is administered parenterally on the same day the ketamine or pharmaceutically acceptable salt thereof is administered, and orally on the day the ketamine or pharmaceutically acceptable salt thereof is not administered. When co-administered, the S-adenosylmethionine can precede the ketamine administration, or the ketamine can precede the S-adenosylmethionine administration. When co-administered in a fixed dose combination, the two active components are administered together.

The ratio of NMDA receptor antagonist to S-adenosylmethionine may vary. In some embodiments, the weight ratio of the NMDA receptor antagonist to S-adenosylmethionine may be from about 1:500, about 1:450, about 1:400, about 1:350, about 1:300, about 1:250, about 1:200, about 1:150, about 1:100, about 1:90, about 1:80, about 1:75, about 1:70, about 1:60, about 1:50, about 1:45, about 1:40, about 1:35, about 1:30, about 1:25, about 1:20, about 1:15, about 1:10, about 1:9, about 1:8, about 1:7, about 1:6, about 1:5, about 1:4, about 1:3, about 1:2, or about 1:1. In some embodiments, the weight ratio of the NMDA receptor antagonist to S-adenosylmethionine can be about 0.001, about 0.002, about 0.003, about 0.004, about 0.005, about 0.01, about 0.05, about 0.1, about 0.2, about 0.3, about 0.4, about 0.5, about 0.6, about 0.7, about 0.8, about 0.9, about 1, about 5 or about 10, or any ratio in a range bounded by, or between, any of these values. For example, a ratio of 0.1 indicates that the weight of NMDA receptor antagonist is 1/10 that of S-adenosylmethionine. A ratio of 10 indicates that the weight of NMDA receptor antagonist is 10 times that of S-adenosylmethionine. Where each of the S-adenosylmethionine or NMDA receptor are in the form of a salt, the ratios refer to the weight of the parent compound.

For example, for combinations of ketamine and SAM-e, a weight ratio of ketamine to SAM-e can be from about 2:1 to about 1:600. In some embodiments, combinations of the present disclosure comprise 800 mg ketamine and 400 mg SAM-e. In some embodiments, combinations of the present disclosure comprise 25 mg ketamine and up to 15 g SAM-e. In some embodiments, combinations of the present disclosure comprise 3 mg ketamine and up to 1 g SAM-e.

It will be understood that the specific dose level and frequency of dosage for any subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the condition.

Selection of a therapeutically effective dose will be determined by the skilled artisan considering several factors, which will be known to one of ordinary skill in the art. Such factors include the particular form of the pharmacological agent, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

### NMDA Receptor Antagonist Dosages

The amount of NMDA receptor antagonist (e.g., ketamine) in a therapeutic composition may vary. For example, some compositions may comprise about 0.0001% (w/v) to about 50% (w/v), about 0.01% (w/v) to about 20% (w/v), about 0.01% to about 10% (w/v), about 0.001% (w/v) to about 1% (w/v), about 0.1% (w/v) to about 0.5% (w/v), about 1% (w/v) to about 3% (w/v), about 3% (w/v) to about 5% (w/v), about 5% (w/v) to about 7% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), or about 40% (w/v) to about 50% (w/v) of the NMDA receptor antagonist. Some compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), or about 80% (w/w) to about 90% (w/w) of NMDA receptor antagonist.

In some embodiments, the NMDA receptor antagonist (e.g., ketamine) or a pharmaceutically acceptable salt thereof can be administered as a solution by an intravenous or intramuscular route. For example, a dose of the NMDA receptor antagonist (e.g., ketamine) administered intravenously can range from about 0.1 mg/kg to about 20 mg/kg, about 0.25 mg/kg to about 20 mg/kg, about 0.25 mg/kg to about 15 mg/kg, about 0.25 mg/kg to about 10 mg/kg, about 0.25 mg/kg to about 5 mg/kg, about 0.25 mg/kg to about 2 mg/kg, about 0.25 mg/kg to about 1 mg/kg, 1 mg/kg to about 20 mg/kg, about 1 mg/kg to about 15 mg/kg, about 2 mg/kg to about 15 mg/kg, 1 mg/kg to about 10 mg/kg, about 1 mg/kg to about 9 mg/kg, about 1 mg/kg to about 8 mg/kg, about 1 mg/kg to about 7 mg/kg, about 1 mg/kg to about 6 mg/kg, about 1 mg/kg to about 5 mg/kg, about 1 mg/kg to about 4.5 mg/kg, about 1 mg/kg to about 4 mg/kg, about 1 mg/kg to about 3.5 mg/kg, about 1 mg/kg to about 3 mg/kg, about 1 mg/kg to about 2.5 mg/kg, about 1 mg/kg to about 2 mg/kg, or about 1 mg/kg to about 1.5 mg/kg, based on the mass of the NMDA receptor antagonist. For example, for combinations of ketamine and SAM-e, ketamine can be administered intravenously or intramuscularly at a range of 0.25 mg/kg to 2 mg/kg. Alternatively, ketamine can be administered intravenously or intramuscularly at a dose of 2 mg/kg to about 15 mg/kg.

When administered intravenously, the NMDA receptor antagonist (e.g., ketamine) or pharmaceutically acceptable salt can be administered over a period of time, for example about 1 second to about 48 hours, for example about 1, 5, 10, 15, 20, 25, 30, 35, 40, 45, 50, 55 or 60 minutes, about 5 hours, about 6 hours, about 7 hours, about 8 hours, about 9 hours, about 10 hours, about 24 hours, about 48 hours or any period of time in-between.

Alternatively, the NMDA receptor antagonist (e.g., ketamine) or a pharmaceutically acceptable salt can be administered intravenously or intramuscularly at a rate of about 1 ug/kg/min to about 100 ug/kg/min, about 2 ug/kg/min to about 50 ug/kg/min, about 3 ug/kg/min to about 50 ug/kg/min, about 4 ug/kg/min to about 50 ug/kg/min, about 5 ug/kg/min to about 100 ug/kg/min, about 5 ug/kg/min to about 90 mg/kg/min, about 5 ug/kg/min to about 80 mg/kg/min, about 5 ug/kg/min to about 70 mg/kg/min, about 5 ug/kg/min to about 60 mg/kg/min, or about 5 ug/kg/min to about 50 mg/kg/min. For example, ketamine or a pharmaceutically acceptable salt thereof can be administered at a range of about 4.16-50 ug/kg/min over about 40 to 60 minutes. Alternatively, ketamine or a pharmaceutically acceptable salt thereof can be administered at a range of about is 5-62 ug/kg/min per kilogram per minute over about 4-6 hours.

Alternatively, the NMDA receptor antagonist (e.g., ketamine) or a pharmaceutically acceptable salt thereof can be administered orally. For example, oral NMDA receptor antagonist (e.g., ketamine) or a pharmaceutically acceptable salt thereof can be administered at a daily dose of about 0.1 mg/kg to about 100 mg/kg, for example about 1 mg/kg to about 20 mg/kg, about 1 mg/kg to about 15 mg/kg, about 1 mg/kg to about 12 mg/kg, about 1 mg/kg to about 10 mg/kg, about 5 mg/kg to about 100 mg/kg, about 5 mg/kg to about 90 mg/kg, about 5 mg/kg to about 80 mg/kg, about 5 mg/kg to about 70 mg/kg, about 8 mg/kg to about 70 mg/kg, and the like. For example, the NMDA receptor antagonist (e.g., ketamine) or a pharmaceutically acceptable salt thereof can be administered at a daily dose of about 1 mg/kg to about 12 mg/kg. Alternatively, the NMDA receptor antagonist (e.g., ketamine) or a pharmaceutically acceptable salt thereof can be administered at a daily dose of about 8 mg/kg to about 70 mg/kg.

For example, oral NMDA receptor antagonist (e.g., ketamine) or a pharmaceutically acceptable salt thereof can be administered at a daily dose of about 1-10 mg, about 5-10 mg, about 10-15 mg, about 15-20 mg, about 20-25 mg, about 25-30 mg, about 28-33 mg, about 30-33 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 10-500 mg, about 50-400 mg, about 50-300 mg, about 100-250 mg, about 1-10 mg, about 10-200 mg, about 10-150 mg, about 10-100 mg, about 10-180 mg, about 10-160 mg, about 10-140 mg, about 10-120 mg, about 10-100 mg, about 10-20 mg, about 20-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-160 mg, about 160-180 mg, about 180-200 mg, about 200-220 mg, about 220-240, about 240-250 mg, about 250-260 mg, about 260-280 mg, about 280-300 mg, about 300-350 mg, about 350-400 mg about 400-500 mg, about 500-600 mg, about 600-700 mg, about 700-800 mg, about 800-900 mg, about 900-1,000 mg, or any amount in-between. For example, oral NMDA receptor antagonist (e.g., ketamine) or a pharmaceutically acceptable salt thereof can be administered at a daily dose of about 1 mg, about 5 mg, about 10 mg, about 15 mg, about 20 mg, about 25 mg, about 50 mg, about 100 mg, about 150 mg, about 200 mg, about 250 mg, about 300, about 400 mg, about 500 mg, about 600 mg, about 700 mg, about 800 mg, about 900 mg or about 1,000 mg per day, or any amount in-between, based on the mass of the NMDA receptor antagonist (e.g., ketamine).

Alternatively, the NMDA receptor antagonist (e.g., ketamine) can be administered intranasally, via a nasal spray, nasal drops, nasal mist, and the like. For example, a daily intranasal dose of the NMDA receptor antagonist (e.g., ketamine) or a pharmaceutically acceptable salt thereof can be about 1-10 mg, about 5-10 mg, about 10-15 mg, about 15-20 mg, about 20-25 mg, about 25-30 mg, about 28-33 mg, about 30-33 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 10-500 mg, about 50-400 mg, about 50-300 mg, about 100-250 mg, about 1-10 mg, about 10-200 mg, about 10-150 mg, about 10-100 mg, about 10-180 mg, about 10-160 mg, about 10-140 mg, about 10-120 mg, about 10-100 mg, about 10-20 mg, about 20-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-160 mg, about 160-180 mg, about 180-200 mg, about 200-220 mg, about 220-240, about 240-250 mg, about 250-260 mg, about 260-280 mg, about 280-300 mg, about 300-350 mg, about 350-400 mg, about 400-500 mg, about 500-600 mg, about 600-700 mg, about 700-800 mg, about 800-900 mg, about 900-1,000 mg, or any amount in-between. For example, an intranasal dose of an NMDA receptor antagonist (e.g., ketamine) or a pharmaceutically acceptable salt thereof can be administered at a daily dose of about 1 mg, about 5 mg, about 10 mg, about 15 mg, about 20 mg, about 25 mg, about 50 mg, about 100 mg, about 150 mg, about 200 mg, about 250 mg, about 300, about 400 mg, about 500 mg, about 600 mg, about 700 mg, about 800 mg, about 900 mg or about 1,000 mg per day, or any amount in-between, based on the mass of the NMDA receptor antagonist (e.g., ketamine).

### S-Adenosylmethionine Dosing

The amount of S-adenosylmethionine in a therapeutic composition may vary. For example, some liquid compositions may comprise about 0.0001% (w/v) to about 50% (w/v), about 0.01% (w/v) to about 20% (w/v), about 0.01% to about 10% (w/v), about 0.001% (w/v) to about 1% (w/v), about 0.1% (w/v) to about 0.5% (w/v), about 1% (w/v) to about 3% (w/v), about 3% (w/v) to about 5% (w/v), about 5% (w/v) to about 7% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), or about 40% (w/v) to about 50% (w/v) of the S-adenosylmethionine. Some compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), or about 80% (w/w) to about 90% (w/w) of S-adenosylmethionine.

In some embodiments, the S-adenosylmethionine or a pharmaceutically acceptable salt thereof can be administered as a solution by an intravenous or intramuscular route. For example, a dose of the S-adenosylmethionine administered intravenously can range from about 1 mg/kg to about 20 mg/kg, for example about 1 mg/kg to about 1,000 mg/kg, about 1 mg/kg to about 500 mg/kg, about 1 mg/kg to about 250 mg/kg, about 1 mg/kg to about 100 mg/kg, about 5 mg/kg to about 1,000 mg/kg, about 5 mg/kg to about 500 mg/kg, about 5 mg/kg to about 250 mg/kg, about 5 mg/kg to about 100 mg/kg, about 10 mg/kg to about 1,000 mg/kg, about 10 mg/kg to about 500 mg/kg, about 10 mg/kg to about 250 mg/kg, or about 10 mg/kg to about 100 mg/kg, based on the mass of S-adenosylmethionine.

Alternatively, the S-adenosylmethionine or a pharmaceutically acceptable salt thereof can be administered orally. For example, oral N S-adenosylmethionine or a pharmaceutically acceptable salt thereof can be administered at a daily dose of about 100-200 mg, about 200-400 mg, about 400-600 mg, about 600-800 mg, about 800-1,000 mg, about 1,000-1,200 mg, about 1,200-1,400, about 1,400-1,600 mg, about 1,600-1,800, about 1,800-2,000, about 2,200-2,400, about 2,400-2,600 mg, about 2,600-2,800, about 2,800-3,000, about 3,200-3,400, about 3,400-3,600 mg, about 3,600-3,800, about 4,800-4,000, about 100 mg, about 200 mg, about 300 mg, about 400 mg, about 500 mg, about 600 mg, about 700 mg, about 800 mg, about 900 mg, about 1,000 mg, about 1,100 mg, about 1,200 mg, about 1,300 mg, about 1,400 mg, about 1,500 mg, about 1,600 mg, about 1,700 mg, about 1,800 mg, about 1,900 mg, about 2,000 mg, about 2,100 mg, about 2,200 mg, about 2,300 mg, about 2,400 mg, about 2,500 mg, about 2,600 mg, about 2,700 mg, about 2,800 mg, about 2,900 mg, about 3,000 mg, about 3,100 mg, about 3,200 mg, about 3,300 mg, about 3,400 mg, about 3,500 mg, about 3,600 mg, about 3,700 mg, about 3,800 mg, about 3,900 mg, or about 4,000 mg per day based on the mass of S-adenosylmethionine.

In some embodiments, the S-adenosylmethionine is orally administered at a dose of about 400 to about 3,200 mg per day. In some embodiments, the S-adenosylmethionine is intravenously administered at a dose of about 400 to about 10,000 mg per day.

### Pharmaceutical Compositions

In some embodiments, the NMDA-receptor antagonist (e.g., ketamine) or pharmaceutically acceptable salt thereof and the SAM-e or pharmaceutically acceptable salt thereof are provided in separate pharmaceutical compositions. In some embodiments, the NMDA-receptor antagonist (e.g., ketamine) or pharmaceutically acceptable salt thereof and the SAM-e or pharmaceutically acceptable salt thereof are provided in one pharmaceutical composition comprising both active ingredients, i.e., a fixed dose combination (FDC). In some embodiments, the pharmaceutical composition comprises at least one carrier.

The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. The use of solvents, dispersion media, coatings, antibacterial and antifungal agents, isotonic and absorption delaying agents, and the like for pharmaceutical active substances that are pharmaceutically acceptable as the term is used herein are well known in the art and are preferably inert. Except insofar as any conventional media or agent is incompatible with the active ingredients, its use in therapeutic compositions is contemplated.

The pharmaceutical compositions of the present application can be administered for any of the uses described herein by any suitable means, for example, orally, such as in the form of a capsule, a sublingual capsule, a tablet, a sublingual tablet, a pill, a dragee, a powder, a granule, an emulsion, a solution, a suppository, a syrup, an elixir, or a suspension. The pharmaceutical compositions can also be administered sublingually or buccally.

In some embodiments, the pharmaceutical compositions can be administered parenterally, such as by subcutaneous, intravenous, or intramuscular; nasally, including administration to the nasal membranes, such as by inhalation spray; topically, such as in the form of a cream or ointment; or rectally such as in the form of suppositories; in dosage unit formulations containing non-toxic, pharmaceutically acceptable vehicles or diluents. The present pharmaceutical compositions can, for example, be administered in a form suitable for immediate release or extended release. Immediate release or extended release can be achieved using suitable pharmaceutical compositions, or, particularly in the case of extended release, using devices such as subcutaneous implants or osmotic pumps. The present pharmaceutical compositions can also be administered liposomally.

Pharmaceutical compositions adapted for oral administration may be capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups can comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The present pharmaceutical compositions can also be delivered through the oral cavity by sublingual and/or buccal administration. Molded-tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions in saline which can contain, for example, benzyl alcohol or other suitable preservatives, absorption promoters to enhance bioavailability, and/or other solubilizing or dispersing agents such as those known in the art.

Pharmaceutical compositions adapted for parenteral administration, intravenous administration, include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerin, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Exemplary compositions for parenteral administration include injectable solutions or suspensions which can contain, for example, suitable non-toxic, parenterally acceptable diluents or solvents, such as mannitol, 1,3-butanediol, water, Lactated Ringer's solution, an isotonic sodium chloride solution, or other suitable dispersing or wetting and suspending agents, including synthetic mono- or diglycerides, and fatty acids, including oleic acid, or Cremaphor.

Alternatively, the pharmaceutical compositions as described herein can be administered for rectal administration. Exemplary compositions for rectal administration include suppositories which can contain, for example, a suitable non-irritating excipient, such as cocoa butter, beeswax, synthetic glyceride esters or polyethylene glycols, which are solid at ordinary temperatures, but liquefy and/or dissolve in the rectal cavity to release the drug.

Exemplary compositions for topical administration include a topical carrier such as Plastibase (mineral oil gelled with polyethylene).

### EXAMPLES

The present disclosure may be better understood by reference to the following non-limiting examples, which are presented in order to more fully illustrate the preferred embodiments of the invention. They should in no way be construed to limit the broad scope of the disclosure.

### Example 1: Clinical Trial

The present study evaluates whether treatment of a condition with a combination of ketamine and SAM-e is superior (e.g., additive, synergistic) to treatment to treatment with ketamine monotherapy.

A clinical trial is performed to determine whether treatment with a combination of ketamine and S-adenosylmethionine (SAM-e) attenuates depression symptoms in subjects with a neurological disorder (e.g., Major Depressive Disorder (MDD), Treatment Resistant Depression (TRD), Suicidal Ideation (SI), Substance Use Disorder (SUD), eating Disorders including anorexia nervosa and bulimia, Complex Regional Pain Syndrome (CRPS), Fibromyalgia (FM), Alzheimer's Disease (AD), Attention Deficit Disorder (ADD), Attention Deficit Hyperactive Disorder (ADHD), obesity, Post Traumatic Stress Disorder (PTSD), and Generalized Anxiety Disorder (GAD)). The study also measures safety, tolerability and pharmacokinetics of ketamine and SAM-e in the subjects. The study also evaluates whether treatment of a condition with a combination of ketamine and SAM-e is associated with reduced ketamine-induced hypertension, hepatotoxicity and/or dissociative experiences as compared with treatment of ketamine as monotherapy.

About 40 subjects are randomized into a ketamine treatment arm and ketamine plus SAM-e treatment arms comprising escalating doses of SAM-e. Ketamine is administered parenterally (intravenously or intramuscularly) and SAM-e is administered orally. Patients can remain on standard of care.

### Study Objectives

The primary objective of the study is to determine whether treatment with a combination of ketamine and SAM-e for about 21 days prevents, reduces a likelihood of, attenuates or decreases depression symptoms in patients compared with ketamine monotherapy.

The secondary objective of this study is to determine the safety, tolerability and pharmacokinetics of combination treatment of ketamine and SAM-e in patients with a neurological disorder (e.g., TRD). Specifically, the study evaluates the effect of SAM-e addition to a ketamine regimen on ketamine-induced hypertension, hepatotoxicity and/or dissociative experiences as compared with treatment of ketamine as monotherapy.

### Study Subjects

About 40 participants are enrolled in the study. Participants have a confirmed diagnosis of a neurological disease (e.g., MDD or TRD) and supporting clinical phenotype. Participants must fulfill Diagnostic and Statistical Manual of Mental Disorders (DSM) criteria for their disorder. Depression is at least moderate severity, defined as Clinical Global Impressions Scale (CGI-S) score of >4. Patients have failed at least one prior course of therapy with other standard of care.

### Study Intervention

Ketamine is provided as a concentrated solution for intravenous or intramuscular injection comprising 200 mg/20 mL (10 mg/mL), 500 mg/10 mL (50 mg/mL) or 500 mg/5 ml (100 mg/mL) racemic ketamine hydrochloride (based on the mass of ketamine) in multidose vial (MDV) containing water-for-injection, a solution of 0.9% sodium chloride in water—for injection (saline), or a solution of 5% dextrose in water-for-injection. The solution is diluted to about 2 mg/mL prior to administration. Esketamine (S-ketamine) or Arketamine (R-ketamine) can be used instead of racemic ketamine.

SAM-e is provided as an oral tablet or capsule comprising 200 or 400 mg SAM-e disulfide tosylate (based on the mass of SAM-e). The tablets or capsules further contain at least one of additional excipients such as, but not limited to, cellulose (e.g., microcrystalline cellulose, hydroxypropylcellulose, hydroxypropylmethylcellulose (Hypromellose), silicon dioxide, calcium oxide, calcium chloride, staric acid, ascorbyl palmitate, lactate, folate, B12 and/or other B vitamins, vitamin D, antioxidant vitamins (A, C, and E), polyunsaturated fatty acids (PUFAs) (e.g., eicosapentaenoic acid and docosahexaenoic acid), N-acetylcysteine (NAC), nicotinamide adenine dinucleotide (NAD+), prebiotics, probiotics, and/or essential trace elements (e.g., selenium, zinc, magnesium, copper, iron, and chromium).

Matching placebo for each of ketamine and SAM-e formulations described above are prepared, containing an identical formulation except that the respective active ingredient is not included.

### Study Design

This is a double-blinded placebo-controlled study. Study arms include ketamine plus placebo, and ketamine plus SAM-e in escalating doses as described in Table 1.

Ketamine is administered starting on day 2 according to one of the following alternative routes:
(A) for treating depression (including MDD, TRD), SI, SUD, PTSD or GAD:
   (i) intravenously at a dose of 0.25 mg/kg to 2 mg/kg over a period of 40-60 minutes (4.16 ug/kg/min to 50 ug/kg/min); or
   (ii) intramuscularly at a dose of 0.25 mg/kg to 2 mg/kg;

Ketamine dosing is repeated every other day for a time period of approximately 12-14 days.
(B) for treating CRPS or fibromyalgia:
   (iii) intravenously at a dose of 2 mg/kg to 15 mg/kg over a period of 4-6 hours; (5 ug/kg/min to 62 ug/kg/min); or
   (iv) intramuscularly at a dose of 2 mg/kg to 15 mg/kg;

Ketamine dosing is repeated every other day for a time period of approximately 10 days.

TABLE 1

| Group | Intervention | SAM-e (daily) | Ketamine (daily) |
|---|---|---|---|
| A | Ketamine + PB | — | (i), (ii), (iii) or (iv) |
| B | Ketamine + SAM-e | 400 mg | (i), (ii), (iii) or (iv) |
| C | Ketamine + SAM-e | 800 mg | (i), (ii), (iii) or (iv) |
| D | Ketamine + SAM-e | 1,600 mg | (i), (ii), (iii) or (iv) |
| E | Ketamine + SAM-e | 3,200 mg | (i), (ii), (iii) or (iv) |

Subjects are evaluated for alleviation of depression symptoms according to various scales as described below. Safety, tolerability and PK are measured as described below.

### Study Endpoints

### Primary Endpoint

The primary endpoint is to assess the effect of a combination of ketamine and SAM-e on the following outcomes:
(a) Change in Montgomery-Asberg Depression Rating Scale (MADRS) compared across ketamine-SAM-e, ketamine-PB and PB administrations.
(b) Change in four-item positive symptom subscale of the Brief Psychiatric Rating Scale (BPRS), Clinician-Administered Dissociative States Scale (CADSS) and Visual Analog Scale (VAS) scores from Time 0 to Time 40 minutes compared across ketamine-SAM-e and ketamine-PB administrations.

### Description of Study Instruments

The Structured Clinical Interview for DSM-5 Axis I Disorders (SCID) is a semi-structured interview that provides probe questions as well as follow-up questions to be asked by the clinician to assist in diagnosis. It includes an overview to obtain information about demographics, work, chief complaint, history of present illness, past history, treatment history, and current functioning. The main body of SCID (patient edition) includes 9 modules that are designed to diagnose 51 mental illnesses in all.

The Brief Psychiatric Rating Scale (BPRS) is used to assess acute behavioral changes during the infusions. Four key BPRS items for the positive (+) symptoms of psychosis are used: conceptual disorganization, hallucinatory behavior, suspiciousness, and unusual thought content. Three items representing the negative (−) symptoms of psychosis are also used: blunted affect, emotional withdrawal, and motor retardation.

The Clinician-Administered Dissociative States Scale (CADSS) is used to measure dissociative effects during infusions. The scale includes 19 questions and 8 observer ratings scored from 0 (not at all) to 4 (extremely). The CADSS measures impairment in body perception, environmental perception, time perception, memory impairment, and feelings of unreality.

Visual Analog Scales. These scales are scored in millimeters from the left-hand side of a 100-mm line to a perpendicular mark made by the patient at a point corresponding to the apparent magnitude of the feeling state. Range: 0 (“not at all”) to 100 (“most ever”).

Montgomery-Asberg Depression Rating Scale (MADRS) This is a 10-item instrument used for the evaluation of depressive symptoms in adults and for the assessment of any changes to those symptoms. Each of the 10 items is rated on a scale of 0 to 6, with differing descriptors for each item. These individual item scores are added together to form a total score, which can range between 0 and 60 points. The estimated time to administer this scale is 20 minutes. Inter-rater reliability of the scale is high and scores correlate significantly with those of the HAM-D. On the infusion days a modified MADRS is used that excludes sleep and appetite items.

Quick Inventory of Depressive Symptomatology, Self Report (QIDS-SR): The Quick Inventory of Depressive Symptomatology, Self Report (QIDS-SR) is a 16-item self rated instrument designed to assess the severity of depressive symptoms. The 16 items cover the nine symptom domains of major depression as indicated by the DSM, and are rated on a scale of 0-3. Total scores range from 0 to 27, with ranges of 0-5 (normal), 6-10 (mild), 11-15 (moderate), 16-20 (moderate to severe), and 21+(severe).

Columbia-Suicide Severity Rating Scale (C-SSRS): The Columbia-Suicide Severity Rating Scale (C-SSRS) is a comprehensive, semi-structured interview that uniquely measures the full spectrum of suicidality including passive and active suicidal ideation, suicidal intent as well as suicidal behaviors. Subjects are assessed with the CSSRS by a study clinician.

### Secondary Endpoints

The Secondary endpoint is a composite safety and tolerability profile. Safety is measured by the frequencies of occurrences of the following: Treatment emergent adverse events (TEAEs)≥grade 2 in severity (CTCAE version 5), all Serious Adverse Events (SAEs), and all Adverse events of special interests (AESIs). Safety and tolerability of the combination of ketamine and SAM-e are determined by monitoring Adverse Events (AEs) over approximately 28 days of treatment via patient interviews, patient diary reviews, physical examinations, vital signs including heart rate and blood pressure, and clinical laboratory safety tests, including liver function tests.

The effects of SAM-e addition to a ketamine regimen on ketamine-induced hypertension, hepatotoxicity, and dissociation as compared with treatment of ketamine as monotherapy are evaluated by comparing liver function tests and vital signs between the study arms. Dissociation is evaluated by the CADSS pre- and post-intervention.

The Columbia-Suicide Severity Rating Scale (C-SSRS) is administered pre- and post-intervention.

An additional secondary endpoint is pharmacokinetics (PK) after 14 days of administration of ketamine or ketamine+SAM-e to study participants. The following pharmacokinetic parameters for plasma ketamine and SAM-e are calculated on the appropriate blood samples on days 1, 7 10 and on the last day of treatment.
AUC: area under the concentration-time curve from time zero (time of drug administration) to infinity
AUCₜₐᵤ: The area under the concentration-time curve during a dosing interval (tau) at steady state.
AUCₗₐₛₜ: area under the concentration-time curve from time zero (time of drug administration) to tₗₐₛₜ
AUC₂₄: area under the concentration-time curve from time zero to 24 h on Day 1.
Cₘₐₓ: Maximum observed concentration.
Tₘₐₓ: Time to reach Cₘₐₓ. If the maximum value occurs at more than one time point, Tₘₐₓ is defined as the first time point with this value.
Cₘᵢₙ: Minimum observed concentration.
Tₘᵢₙ: Time to reach Cₘᵢₙ.
tₗₐₛₜ: time corresponding to Clast
t_{1/2,z} (t_{1/2}): terminal elimination half-life of the compound, calculated as:
   t_{1/2,z}=ln(2)/λ_{z} (λ_{z} being the rate constant of the terminal phase).
Rac(AUCτ) (RacAUC): accumulation ratio of AUC (AUC_{τ}/AUC₂₄)
RacCₘₐₓ (RacCₘₐₓ): accumulation ratio of Cₘₐₓ (Cₘₐₓ Dayxx/Cₘₐₓ Day 1)

### Example 2: Clinical Trial

A clinical trial is performed to determine whether treatment with a combination of ketamine and S-adenosylmethionine (SAM-e) attenuates depression symptoms in subjects with a neurological disorder as described in Example 1. About 40 subjects are randomized into a ketamine treatment arm and ketamine plus SAM-e treatment arms. Ketamine is administered parenterally and SAM-e is administered parenterally or orally according to an alternating dosing schedule. Patients can remain on standard of care. The study objectives and endpoints are as described in Example 1.

### Study Intervention

Ketamine is provided as a concentrated solution for intravenous or intramuscular injection as described in Example 1.

SAM-e is provided as:
(a) A powder for injection. Just before administration, SAM-e is dissolved in water-for-injection, saline, 5% dextrose solution, or lactated Ringers solution at a concentration of 5 mg/ml in a 1000 ml intravenous solution bag). SAM-e solution for injection is prepared in proximity to administration, and administered over a prescribed time via a programmable syringe pump or infusion pump.
(b) Oral tablet or capsule comprising 400 mg SAM-e disulfate tosylate (based on the mass of SAM-e), as described in Example 1.

Matching placebo for each of ketamine and SAM-e formulations described above are prepared, containing an identical formulation except that the respective active ingredient is not included.

### Study Design

This is a double-blinded placebo-controlled study. Study arms include ketamine plus placebo, and ketamine plus SAM-e oral or intravenous in escalating doses as described in Table 2.

SAM-e is administered according to an alternating schedule. On day 1, SAM-e is orally administered at a dose of 400 mg to 3,200 mg. On day 2, SAM-e is intravenously or intramuscularly administered at a dose of 10 mg/kg to 250 mg/Kg. Starting on day 3, SAM-e is administered according to an alternating dosing regimen (oral and i.v./i.m.) for a total of 14 days as described in Table 2.

Ketamine is administered starting on day 2 intravenously or intramuscularly according to the schedule described in Example 1.

On the days that ketamine and SAM-e are administered parenterally, SAM-e is administered prior to Ketamine.

TABLE 2

| | | | Ketamine |
|---|---|---|---|
| | | SAM-e | D2, 4, 6, 8, 10, 12, |
| | | Oral: D1, 3, 5, 7, 9, | 14 |
| | | 11, 13 | (regimens are |
| | | i.v./i.m. D2, 4, 6, 8, | described in |
| Group | Intervention | 10, 12, 14 | Example 1) |
| A | Ketamine + PB | — | (i), (ii), (iii) or (iv) |
| B | Ketamine + SAM-e | 400 mg | (i), (ii), (iii) or (iv) |
| | | i.v./i.m. 10 mg/kg | |
| C | Ketamine + SAM-e | 800 mg | (i), (ii), (iii) or (iv) |
| | | i.v./i.m. 75 mg/kg | |
| D | Ketamine + SAM-e | 1,600 mg | (i), (ii), (iii) or (iv) |
| | | i.v./i.m. 125 mg/kg | |
| E | Ketamine + SAM-e | 3,200 mg | (i), (ii), (iii) or (iv) |
| | | i.v./i.m. 250 mg/kg | |

Subjects are evaluated for alleviation of depression symptoms according to various scales as described below. Safety, tolerability and PK are measured as described in Example 1.

### Example 3: Clinical Trial

A clinical trial is performed to determine whether treatment with a combination of ketamine and S-adenosylmethionine (SAM-e) attenuates depression symptoms in subjects with a neurological disorder as described in Example 1. About 40 subjects are randomized into a ketamine treatment arm and ketamine plus SAM-e treatment arms. Ketamine and SAM-e are each administered orally. Patients can remain on standard of care. The study objectives and endpoints are as described in Example 1.

### Study Intervention

Ketamine is provided as an oral tablet or capsule comprising 200 mg racemic ketamine hydrochloride (based on the mass of ketamine). Esketamine (S-ketamine) or Arketamine (R-ketamine) can be used instead of racemic ketamine.

SAM-e is provided as an oral tablet or capsule comprising 400 mg SAM-e disulfate tosylate (based on the mass of SAM-e), as described in Example 1. The tablets or capsules can contain additional excipients as described in Example 1.

Matching placebo for each of ketamine and SAM-e formulations described above are prepared, containing an identical formulation except that the respective active ingredient is not included.

### Study Design

This is a double-blinded placebo-controlled study. Study arms ketamine plus placebo, and ketamine plus SAM-e in escalating doses as described in Table 3. Ketamine and SAM-e are each administered daily for approximately 12-14 days.

TABLE 3

| Group | Intervention | SAM-e (daily) | Ketamine (daily) |
|---|---|---|---|
| A | Ketamine + PB | — | 200 mg |
| B | Ketamine + SAM-e | 400 mg | 200 mg |
| C | Ketamine + SAM-e | 800 mg | 200 mg |
| D | Ketamine + SAM-e | 1,600 mg | 200 mg |
| E | Ketamine + SAM-e | 3,200 mg | 200 mg |

Subjects are evaluated for alleviation of depression symptoms according to various scales as described below. Safety, tolerability and PK are measured as described in Example 1.

### Example 4: Clinical Trial

A clinical trial is performed to determine whether treatment with a combination of ketamine and S-adenosylmethionine (SAM-e) attenuates depression symptoms in subjects with a neurological disorder as described in Example 1. About 40 subjects are randomized into a ketamine treatment arm and ketamine plus SAM-e treatment arms. Ketamine and SAM-e are co-formulated for oral administration. Patients can remain on standard of care. The study objectives and endpoints are as described in Example 1.

### Study Intervention

Ketamine and SAM-e are co-formulated into oral tablet or capsule (KS) comprising 200 mg racemic ketamine hydrochloride (based on the mass of ketamine) and 400 mg SAM-e disulfate tosylate (based on the mass of SAM-e). Esketamine (S-ketamine) or Arketamine (R-ketamine) can be used instead of racemic ketamine The tablets or capsules can contain additional excipients as described in Example 1.

Matching placebo tablets or capsules are prepared, containing an identical formulation except that one or both of the active ingredients are not included: (a) placebo 1 comprises no active ingredients (PB); (b) placebo 2 comprises ketamine and no SAM-e (SPB); and (c) placebo 3 comprises SAM-e and no ketamine (KPB). Ketamine nasal spray (Spravato) (NS) is also used as a comparator. A matching placebo nasal spray (PBNS) is prepared, containing an identical formulation except that the ketamine active ingredient is not included.

### Study Design

This is a double-blind placebo-controlled study. Study arms include (A) PB with NS, (B) SPB with PBNS, and either (C)/(D) KS and PBNS; or (E)/(F) NS and KPB or with SAM-e in escalating doses as described in Table 4. Ketamine and SAM-e are co-formulated and administered daily for approximately 12-21 days.

TABLE 4

| Group | Intervention | SAM-e | Ketamine |
|---|---|---|---|
| A | PB + NS | — | 28 mg |
| B | SPB + PBNS | — | 200 mg |
| C | Ketamine + SAM-e (KS) + PBNS | 800 mg | 200 mg |
| D | Ketamine + SAM-e (KS) + PBNS | 3200 mg | 200 mg |
| E | KPB + NS | 800 mg | 28 mg |
| F | KPB + NS | 3,200 mg | 28 mg |

Subjects are evaluated for alleviation of depression symptoms according to various scales as described below. Safety, tolerability and PK are measured as described in Example 1.

The present disclosure is not to be limited in scope by the specific embodiments described herein. Indeed, various modifications of the invention in addition to those described herein will become apparent to those skilled in the art from the foregoing description and the accompanying FIGURES. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

### Exemplary Compositions, Methods and Uses are Set Out in the Following Embodiments

Embodiment 1. A combination comprising:
(i) a first pharmaceutical composition comprising in a unit dosage form an N-methyl-D-aspartate (NMDA) receptor antagonist or a pharmaceutically acceptable salt thereof and a pharmaceutically acceptable carrier or excipient; and
(ii) a second pharmaceutical composition comprising in a solid unit dosage form S-adenosylmethionine or a pharmaceutically acceptable salt thereof, and a pharmaceutically acceptable carrier or excipient.

Embodiment 2. The combination of embodiment 1, wherein the NMDA receptor antagonist is selected from the group consisting of ketamine, dextromethorphan, memantine, amantadine, neramexane, phenylcyclidine, and pharmaceutically acceptable salts thereof.

Embodiment 3. The combination of embodiment 1 or 2, wherein the NMDA receptor antagonist is ketamine or a pharmaceutically acceptable salt thereof.

Embodiment 4. The combination of embodiment 3, wherein the ketamine is racemic ketamine or a pharmaceutically acceptable salt thereof.

Embodiment 5. The combination of embodiment 3, wherein the ketamine is esketamine (S-ketamine) or a pharmaceutically acceptable salt thereof.

Embodiment 6. The combination of embodiment 3, wherein the ketamine is Arketamine (R-ketamine) or a pharmaceutically acceptable salt thereof.

Embodiment 7. The combination of any one of embodiments 1-6, wherein the first pharmaceutical composition comprises a pharmaceutically acceptable salt of ketamine.

Embodiment 8. The combination of embodiment 7, wherein the pharmaceutically acceptable salt is a hydrochloride salt.

Embodiment 9. The combination of embodiment 1, wherein the second pharmaceutical composition comprises a pharmaceutically acceptable salt of S-adenosylmethionine.

Embodiment 10. The combination of embodiment 9, wherein the pharmaceutically acceptable salt is S-adenosylmethionine disulfate tosylate, S-adenosylmethionine 1,4-butanedisulfonate or S-adenosylmethionine phytate.

Embodiment 11. The combination of embodiment 1, wherein the first pharmaceutical composition is suitable for parenteral, intravenous, intramuscular, subcutaneous, intranasal, transdermal, sublingual, buccal, or oral administration.

Embodiment 12. The combination of embodiment 1, wherein the first pharmaceutical composition is in a solid dosage form.

Embodiment 13. The combination of embodiment 1, wherein the first pharmaceutical composition is in a liquid dosage form.

Embodiment 14. The combination of embodiment 1, wherein the first pharmaceutical composition is in a form selected from the group consisting of a capsule, a sublingual capsule, a tablet, a sublingual tablet, a pill, a dragee, a powder, a granule, an emulsion, a solution, a suppository, a syrup, an elixir, and a suspension.

Embodiment 15. The combination of embodiment 1, wherein the first pharmaceutical composition is in the form of a solution or suspension, the solution or suspension comprising from about 1 mg/ml to about 100 mg/ml ketamine or a pharmaceutically acceptable salt thereof based on the mass of ketamine.

Embodiment 16. The combination of embodiment 15, wherein the solution or suspension comprises from about 10 mg/ml to about 100 mg/ml ketamine or a pharmaceutically acceptable salt thereof based on the mass of ketamine.

Embodiment 17. The combination of embodiment 1, wherein the second pharmaceutical composition is provided in the form of a lyophilized powder.

Embodiment 18. The combination of embodiment 17, wherein the lyophilized powder is suitable, after reconstitution in an aqueous vehicle, for parenteral, intravenous, intramuscular, or subcutaneous administration.

Embodiment 19. The combination of embodiment 1, wherein the second pharmaceutical composition is in a form suitable for parenteral, intravenous, intramuscular, subcutaneous, intranasal, transdermal, sublingual, buccal, or oral administration.

Embodiment 20. The combination of embodiment 1, wherein the second pharmaceutical composition is in a form selected from the group consisting of a capsule, a sublingual capsule, a tablet, a sublingual tablet, a pill, a dragee, a powder, and a granule.

Embodiment 21. The combination of embodiment 1, wherein the second pharmaceutical composition is formulated for oral administration and comprises from about 400 mg to about 3,200 mg S-adenosylmethionine or the pharmaceutically acceptable salt thereof.

Embodiment 22. The combination of embodiment 1, wherein the second pharmaceutical composition is formulated for parenteral administration and comprises from about 1 mg to about 15 g S-adenosylmethionine or the pharmaceutically acceptable salt thereof.

Embodiment 23. The combination of embodiment 1, wherein the second pharmaceutical composition is included in a kit, the kit comprising the S-adenosylmethionine or pharmaceutically acceptable salt thereof in a powder form, and a separate container comprising an aqueous vehicle for reconstitution.

Embodiment 24. The combination of embodiment 1, wherein the powder form is mixed with the aqueous vehicle to form a solution or suspension comprising from about 1 mg/ml to about 100 mg/ml S-adenosylmethionine or a pharmaceutically acceptable salt thereof based on the mass of S-adenosylmethionine.

Embodiment 25. The combination of embodiment 24, wherein the powder form is mixed with the aqueous vehicle to form a solution or suspension comprising about 5 mg/ml S-adenosylmethionine or a pharmaceutically acceptable salt thereof based on the mass of S-adenosylmethionine.

Embodiment 26. The combination of embodiment 1, wherein the NMDA receptor antagonist or pharmaceutically acceptable salt thereof and the S-adenosylmethionine or pharmaceutically acceptable salt thereof are co-formulated in a fixed dose combination.

Embodiment 27. A fixed dose combination comprising, in unit dosage form:
(i) an N-methyl-D-aspartate (NMDA) receptor antagonist or a pharmaceutically acceptable salt thereof;
(ii) S-adenosylmethionine or a pharmaceutically acceptable salt thereof; and
(iii) and a pharmaceutically acceptable carrier or excipient.

Embodiment 28. A method of treating a neurological condition in a subject in need thereof, the method comprising administering to the subject a combination comprising a therapeutically effective amount of an N-methyl-D-aspartate (NMDA) receptor antagonist or a pharmaceutically acceptable salt thereof, and a therapeutically effective amount of S-adenosylmethionine or a pharmaceutically acceptable salt thereof.

Embodiment 29. The method of embodiment 28, wherein the neurological condition is selected from the group consisting of affective disorders, psychiatric disorders, cerebral function disorders, movement disorders, dementias, motor neuron diseases, neurodegenerative disease, seizure disorders, and headaches.

Embodiment 30. The method of embodiment 20, wherein the neurological condition is selected from the group consisting of Major Depressive Disorder (MDD), Treatment Resistant Depression (TRD), Suicidal Ideation (SI), Substance Use Disorder (SUD), Complex Regional Pain Syndrome (CRPS), Fibromyalgia (FM), Alzheimer's Disease (AD), Attention Deficit Disorder (ADD), Attention Deficit Hyperactive Disorder (ADHD), obesity, Post Traumatic Stress Disorder (PTSD), and Generalized Anxiety Disorder (GAD).

Embodiment 31. The method of any one of embodiments 28 to 30, wherein administering the combination results in reduced hypertension in the subject as compared with hypertension measured in the subject after receiving the NMDA receptor antagonist or pharmaceutically acceptable salt thereof as monotherapy.

Embodiment 32. The method of any one of embodiments 28 to 30, wherein administering the combination results in reduced hepatotoxicity in the subject as compared with hepatotoxicity measured in the subject after receiving the NMDA receptor antagonist or pharmaceutically acceptable salt thereof as monotherapy.

Embodiment 33. The method of any one of embodiments 28 to 30, wherein administering the combination results in reduced dissociative effects in the subject compared with dissociation measured in the subject after receiving the NMDA receptor antagonist or pharmaceutically acceptable salt thereof as monotherapy.

Embodiment 34. A method of reducing hypertension in a subject receiving an N-methyl-D-aspartate (NMDA) receptor antagonist or a pharmaceutically acceptable salt thereof to treat a neurological condition, the method comprising administering to the subject a combination comprising a therapeutically effective amount of the NMDA receptor antagonist or pharmaceutically acceptable salt thereof, and a therapeutically effective amount of S-adenosylmethionine or a pharmaceutically acceptable salt thereof.

Embodiment 35. A method of reducing hepatotoxicity in a subject receiving an N-methyl-D-aspartate (NMDA) receptor antagonist or a pharmaceutically acceptable salt thereof to treat a neurological condition, the method comprising administering to the subject a combination comprising a therapeutically effective amount of the NMDA receptor antagonist or pharmaceutically acceptable salt thereof, and a therapeutically effective amount of S-adenosylmethionine or a pharmaceutically acceptable salt thereof.

Embodiment 36. A method of reducing a dissociative effect in a subject receiving an N-methyl-D-aspartate (NMDA) receptor antagonist or a pharmaceutically acceptable salt thereof to treat a neurological condition, the method comprising administering to the subject a combination comprising a therapeutically effective amount of the NMDA receptor antagonist or pharmaceutically acceptable salt thereof, and a therapeutically effective amount of S-adenosylmethionine or a pharmaceutically acceptable salt thereof.

Embodiment 37. The method of any one of embodiments 20 to 36, wherein the NMDA receptor antagonist or pharmaceutically acceptable salt thereof and the S-adenosylmethionine or pharmaceutically acceptable salt thereof are administered concurrently.

Embodiment 38. The method of any one of embodiments 20 to 36 wherein the NMDA receptor antagonist or pharmaceutically acceptable salt thereof and the S-adenosylmethionine or pharmaceutically acceptable salt thereof are administered sequentially.

Embodiment 39. The method of embodiment 38, wherein the S-adenosylmethionine or pharmaceutically acceptable salt thereof is administered first.

Embodiment 40. The method of embodiment 38, wherein the NMDA receptor antagonist or pharmaceutically acceptable salt thereof is administered first.

Embodiment 41. The method of any one of embodiments 20 to 36, wherein the combination effectuates a therapeutic effect in the subject that is synergistic compared to a therapeutic effect of each component of the combination.

Embodiment 42. The method of any one of embodiments 20 to 36, wherein the combination effectuates a therapeutic effect in the subject that is additive compared to a therapeutic effect of each component of the combination.

Embodiment 43. The method of any one of embodiments 20 to 36, wherein
(i) the NMDA receptor antagonist or pharmaceutically acceptable salt thereof is administered in a first pharmaceutical composition in unit dosage form, the unit dosage form further comprising a pharmaceutically acceptable carrier or excipient; and
(ii) the S-adenosylmethionine or pharmaceutically acceptable salt thereof is administered in a second pharmaceutical composition in a unit dosage form, the unit dosage form further comprising a pharmaceutically acceptable carrier or excipient.

Embodiment 44. The method of any one of embodiments 20 to 36, wherein the NMDA receptor antagonist or pharmaceutically acceptable salt thereof and the S-adenosylmethionine or pharmaceutically acceptable salt thereof are co-formulated in a fixed dose combination.

Embodiment 45. The method of any one of embodiments 20 to 36, wherein the NMDA receptor antagonist is administered parenterally, intravenously, intramuscularly, subcutaneously, intranasally, transdermally, sublingually, buccally, or orally.

Embodiment 46. The method of any one of embodiments 20 to 36, wherein the S-adenosylmethionine or pharmaceutically acceptable salt thereof is administered parenterally, intravenously, intramuscularly, subcutaneously, intranasally, transdermally, sublingually, buccally, or orally.

Embodiment 47. The method of any one of embodiments 20 to 36, wherein the NMDA receptor antagonist or pharmaceutically acceptable salt thereof and the S-adenosylmethionine or pharmaceutically acceptable salt thereof are each administered parenterally.

Embodiment 48. The method of any one of embodiments 20 to 36, wherein the NMDA receptor antagonist or pharmaceutically acceptable salt thereof and the S-adenosylmethionine or pharmaceutically acceptable salt thereof are each administered orally.

Embodiment 49. The method of any one of embodiments 20 to 36, wherein the NMDA receptor antagonist or pharmaceutically acceptable salt thereof is administered parenterally, and the S-adenosylmethionine or pharmaceutically acceptable salt thereof is administered orally.

Embodiment 50. The method of any one of embodiments 20 to 36, wherein the NMDA receptor antagonist or pharmaceutically acceptable salt thereof is administered orally, and the S-adenosylmethionine or pharmaceutically acceptable salt thereof is administered parenterally.

Embodiment 51. The method of any one of embodiments 28 to 36, wherein the NMDA receptor antagonist is selected from the group consisting of ketamine, dextromethorphan, memantine, amantadine, neramexane, phenylcyclidine, and pharmaceutically acceptable salts thereof.

Embodiment 52. The method of embodiment 51, wherein the NMDA receptor antagonist is ketamine or a pharmaceutically acceptable salt thereof.

Embodiment 53. The method of embodiment 38, comprising intravenously administering from about 0.25 mg/kg to about 2 mg/kg of the ketamine or pharmaceutically acceptable salt over a time period.

Embodiment 54. The method of embodiment 53, wherein the time period is about 40 minutes to about 60 minutes.

Embodiment 55. The method of embodiment 52, comprising intramuscularly administering about 2 mg/kg to about 15 mg/kg of the ketamine or pharmaceutically acceptable salt thereof over a time period.

Embodiment 56. The method of embodiment 55, wherein the time period is about 4 hours to about 6 hours.

Embodiment 57. The method of embodiment 38, wherein the ketamine or pharmaceutically acceptable salt thereof is administered every other day for a time period.

Embodiment 58. The method of embodiment 40, wherein the time period is at least about 10 to about 21 days.

Embodiment 59. The method of embodiment 52, wherein the ketamine or pharmaceutically acceptable salt thereof and the S-adenosylmethionine or pharmaceutically acceptable salt thereof are each administered intravenously.

Embodiment 60. The method of embodiment 38, wherein the ketamine or pharmaceutically acceptable salt thereof and the S-adenosylmethionine or pharmaceutically acceptable salt thereof are each administered orally.

Embodiment 61. The method of embodiment 38, wherein the ketamine or pharmaceutically acceptable salt thereof is administered intravenously, and the S-adenosylmethionine or pharmaceutically acceptable salt thereof is administered orally.

Embodiment 62. The method of embodiment 38, wherein the ketamine or pharmaceutically acceptable salt thereof is administered orally, and the S-adenosylmethionine or pharmaceutically acceptable salt thereof is administered intravenously.

Embodiment 63. The method of embodiment 52, wherein the ketamine or pharmaceutically acceptable salt thereof and the S-adenosylmethionine or pharmaceutically acceptable salt thereof are administered in a fixed dose combination.

Embodiment 64. The method of embodiment 38, wherein
the ketamine or pharmaceutically acceptable salt thereof is administered intravenously every other day; and
the S-adenosylmethionine or pharmaceutically acceptable salt thereof is administered daily.

Embodiment 65. The method of embodiment 64, wherein the S-adenosylmethionine or pharmaceutically acceptable salt thereof is administered orally.

Embodiment 66. The method of embodiment 64, wherein the S-adenosylmethionine or pharmaceutically acceptable salt thereof is administered orally or intravenously on alternating days.

Embodiment 67. The method of embodiment 66, wherein the S-adenosylmethionine or pharmaceutically acceptable salt thereof is administered intravenously on the same day the ketamine or pharmaceutically acceptable salt thereof is administered, and orally on the day the ketamine or pharmaceutically acceptable salt thereof is not administered.

Embodiment 68. The method of embodiment 52, wherein the ketamine or pharmaceutically acceptable salt and the S-adenosylmethionine or pharmaceutically acceptable salt thereof are each administered orally, wherein the administration is daily.

Embodiment 69. The method of any one of embodiments 28 to 68, wherein the S-adenosylmethionine is orally administered at a dose of about 400 to about 3,200 mg per day.

Embodiment 70. The method of any one of embodiments 28 to 68, wherein the S-adenosylmethionine is intravenously administered at a dose of about 400 to about 10,000 mg per day.

What is claimed is:

1. A combination comprising:
(i) a first pharmaceutical composition comprising in a unit dosage form an N-methyl-D-aspartate (NMDA) receptor antagonist or a pharmaceutically acceptable salt thereof and a pharmaceutically acceptable carrier or excipient; and
(ii) a second pharmaceutical composition comprising in a solid unit dosage form S-adenosylmethionine or a pharmaceutically acceptable salt thereof, and a pharmaceutically acceptable carrier or excipient.

2. The combination of claim 1, wherein the NMDA receptor antagonist is selected from the group consisting of ketamine, dextromethorphan, memantine, amantadine, neramexane, phenylcyclidine, and pharmaceutically acceptable salts thereof.

3. The combination of claim 1, wherein the NMDA receptor antagonist is ketamine or a pharmaceutically acceptable salt thereof.

4. The combination of claim 3, wherein the ketamine is racemic ketamine or a pharmaceutically acceptable salt thereof.

5. The combination of claim 3, wherein the ketamine is esketamine (S-ketamine) or a pharmaceutically acceptable salt thereof.

6. The combination of claim 3, wherein the ketamine is Arketamine (R-ketamine) or a pharmaceutically acceptable salt thereof.

7. The combination of claim 1, wherein the first pharmaceutical composition comprises a pharmaceutically acceptable salt of ketamine.

8. The combination of claim 7, wherein the pharmaceutically acceptable salt is a hydrochloride salt.

9. The combination of claim 1, wherein the second pharmaceutical composition comprises a pharmaceutically acceptable salt of S-adenosylmethionine.

10. The combination of claim 9, wherein the pharmaceutically acceptable salt is S-adenosylmethionine disulfate tosylate, S-adenosylmethionine phytate, or S-adenosylmethionine 1,4-butanedisulfonate.

11. The combination of claim 1, wherein the second pharmaceutical composition is formulated for oral administration and comprises from about 400 mg to about 3,200 mg S-adenosylmethionine or the pharmaceutically acceptable salt thereof.

12. The combination of claim 1, wherein the second pharmaceutical composition is formulated for parenteral administration and comprises from about 1 mg to about 15 g S-adenosylmethionine or the pharmaceutically acceptable salt thereof.

13. The combination of claim 1, wherein NMDA receptor antagonist or pharmaceutically acceptable salt thereof and the S-adenosylmethionine or pharmaceutically acceptable salt thereof are co-formulated in a fixed dose combination.

14. A fixed dose combination comprising, in unit dosage form:
(i) an N-methyl-D-aspartate (NMDA) receptor antagonist or a pharmaceutically acceptable salt thereof;
(ii) S-adenosylmethionine or a pharmaceutically acceptable salt thereof; and
(iii) and a pharmaceutically acceptable carrier or excipient.

15. A method of treating a neurological condition in a subject in need thereof, the method comprising administering to the subject a combination comprising a therapeutically effective amount of an N-methyl-D-aspartate (NMDA) receptor antagonist or a pharmaceutically acceptable salt thereof, and a therapeutically effective amount of S-adenosylmethionine or a pharmaceutically acceptable salt thereof.

16. The method of claim 15, wherein the neurological condition is selected from the group consisting of affective disorders, psychiatric disorders, cerebral function disorders, movement disorders, dementias, motor neuron diseases, neurodegenerative disease, seizure disorders, and headaches.

17. The method of claim 15, wherein the neurological condition is selected from the group consisting of Major Depressive Disorder (MDD), Treatment Resistant Depression (TRD), Suicidal Ideation (SI), Substance Use Disorder (SUD), Complex Regional Pain Syndrome (CRPS), Fibromyalgia (FM), Alzheimer's Disease (AD), Attention Deficit Disorder (ADD), Attention Deficit Hyperactive Disorder (ADHD), obesity, Post Traumatic Stress Disorder (PTSD), and Generalized Anxiety Disorder (GAD).

18. The method of claim 15, wherein administering the combination results in reduced hypertension in the subject as compared with hypertension measured in the subject after receiving the NMDA receptor antagonist or pharmaceutically acceptable salt thereof as monotherapy.

19. The method of claim 15, wherein administering the combination results in reduced hepatotoxicity in the subject as compared with hepatotoxicity measured in the subject after receiving the NMDA receptor antagonist or pharmaceutically acceptable salt thereof as monotherapy.

20. The method of claim 15, wherein administering the combination results in reduced dissociative effects in the subject compared with dissociation measured in the subject after receiving the NMDA receptor antagonist or pharmaceutically acceptable salt thereof as monotherapy.

21. The method of claim 15, wherein the combination effectuates a therapeutic effect in the subject that is synergistic compared to a therapeutic effect of each component of the combination.

22. The method of claim 15, wherein the combination effectuates a therapeutic effect in the subject that is additive compared to a therapeutic effect of each component of the combination.

23. The method of claim 15, wherein
(i) the NMDA receptor antagonist or pharmaceutically acceptable salt thereof is administered in a first pharmaceutical composition in unit dosage form, the unit dosage form further comprising a pharmaceutically acceptable carrier or excipient; and
(ii) the S-adenosylmethionine or pharmaceutically acceptable salt thereof is administered in a second pharmaceutical composition in a unit dosage form, the unit dosage form further comprising a pharmaceutically acceptable carrier or excipient.

24. The method of claim 15, wherein the NMDA receptor antagonist or pharmaceutically acceptable salt thereof and the S-adenosylmethionine or pharmaceutically acceptable salt thereof are co-formulated in a fixed dose combination.

25. The method of claim 15, wherein the NMDA receptor antagonist or pharmaceutically acceptable salt thereof and the S-adenosylmethionine or pharmaceutically acceptable salt thereof are each administered parenterally.

26. The method of claim 15, wherein the NMDA receptor antagonist or pharmaceutically acceptable salt thereof and the S-adenosylmethionine or pharmaceutically acceptable salt thereof are each administered orally.

27. The method of claim 15, wherein the NMDA receptor antagonist or pharmaceutically acceptable salt thereof is administered parenterally, and the S-adenosylmethionine or pharmaceutically acceptable salt thereof is administered orally.

28. The method of claim 15, wherein the NMDA receptor antagonist or pharmaceutically acceptable salt thereof is administered orally, and the S-adenosylmethionine or pharmaceutically acceptable salt thereof is administered parenterally.

29. The method of claim 15, wherein the NMDA receptor antagonist is selected from the group consisting of ketamine, dextromethorphan, memantine, amantadine, neramexane, phenylcyclidine, and pharmaceutically acceptable salts thereof.

30. The method of claim 29, wherein the NMDA receptor antagonist is ketamine or a pharmaceutically acceptable salt thereof.

31. The method of claim 30, comprising intravenously administering from about 0.25 mg/kg to about 2 mg/kg of the ketamine or pharmaceutically acceptable salt over a time period of about 40 minutes to about 60 minutes.

32. The method of claim 30, comprising intravenously administering from about 2 mg/kg to about 15 mg/kg of the ketamine or pharmaceutically acceptable salt over a time period of about 4 hours to about 6 hours.

33. The method of claim 30 wherein the ketamine or pharmaceutically acceptable salt thereof is administered every other day for a time period of at least about 10 to about 21 days.

34. The method of claim 30, wherein
the ketamine or pharmaceutically acceptable salt thereof is administered intravenously every other day; and
the S-adenosylmethionine or pharmaceutically acceptable salt thereof is administered daily.

35. The method of claim 34, wherein the S-adenosylmethionine or pharmaceutically acceptable salt thereof is administered orally.

36. The method of claim 34, wherein the S-adenosylmethionine or pharmaceutically acceptable salt thereof is administered intravenously.

37. The method of claim 34, wherein the S-adenosylmethionine or pharmaceutically acceptable salt thereof is administered intravenously on the same day the ketamine or pharmaceutically acceptable salt thereof is administered, and orally on the day the ketamine or pharmaceutically acceptable salt thereof is not administered.

38. The method of claim 30, wherein the ketamine or pharmaceutically acceptable salt and the S-adenosylmethionine or pharmaceutically acceptable salt thereof are each administered orally, wherein the administration is daily.

39. The method of claim 15, wherein the S-adenosylmethionine is orally administered at a dose of about 400 to about 3,200 mg per day.

40. The method of claim 15, wherein the S-adenosylmethionine is intravenously administered at a dose of about 400 to about 10,000 mg per day.
